(19) United States
(12) Patent Application Publication
(10) Pub. No.: US 2013/0156755 A1
(43) Pub. Date: Jun. 20, 2013

(54) CANCER THERAPY USING A COMBINATION OF A HSP90 INHIBITORY COMPOUNDS AND A VEGF INHIBITOR

(71) Applicants: Ronald K. Blackman, Brookline, MA (US); Kevin Paul Foley, Landsdale, PA (US); David Proia, Newton, MA (US)

(72) Inventors: Ronald K. Blackman, Brookline, MA (US); Kevin Paul Foley, Landsdale, PA (US); David Proia, Newton, MA (US)

(73) Assignee: Synta Pharmaceuticals Corp., Lexington, MA (US)

(21) Appl. No.: 13/642,064

(22) PCT Filed: Apr. 19, 2011
(86) PCT No.: PCT/US11/33010

§ 371 Date: Feb. 22, 2013

Related U.S. Application Data
(60) Provisional application No. 61/342,827, filed on Apr. 19, 2010.

(51) Int. Cl. A61K 39/395; A61K 31/4196

(52) U.S. Cl. CPC A61K 39/3955; A61K 31/4196
USPC 424/133.1; 514/384; 514/383

(57) ABSTRACT

A pharmaceutical combination comprising a VEGF inhibitor, and an Hsp90 inhibitor according to the following formulae (I) or (Ia) a tautomer, or a pharmaceutically acceptable salt thereof, wherein the variables in the structural formulae are defined herein. Also provided are methods for treating a proliferative disorder in a subject in need thereof, using pharmaceutical combinations described herein.

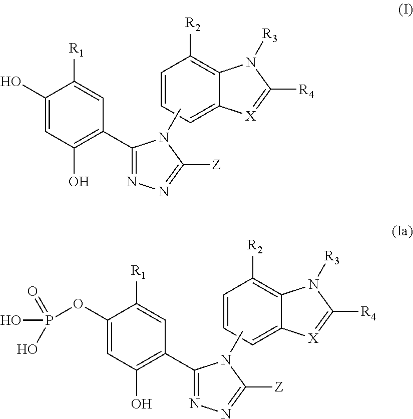

### CROSS-REFERENCE TO RELATED PATENT APPLICATIONS

This application claims the benefit of U.S. Provisional Application No. 61/342,827, filed on Apr. 19, 2010, the entire disclosure of which is incorporated herein by reference.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that certain triazolone Hsp90 inhibitors and VEGF inhibitor combinations are surprisingly effective at treating subjects with cancer and/or angiogenesis/neovascularization diseases without further increasing the side effect profile of the single agents. The particular combination therapies disclosed herein demonstrate surprising biological activity by demonstrating significant anticancer effects.

The present method utilizes Hsp90 inhibitors according to formulae (I) or (Ia), or a compound in Tables 1 or 2 for the treatment of proliferative disorders, such as cancer, in combination with a VEGF inhibitor. A method of treating a subject with cancer includes the step of administering to the subject an Hsp90 inhibitor according to formulae (I) or (Ia), or a compound in Tables 1 or 2 and a VEGF inhibitor useful for the treatment of cancer. In one embodiment, the administration of the Hsp90 inhibitor and the VEGF inhibitor are done concurrently. In another embodiment, the administration of the Hsp90 inhibitor and the VEGF inhibitor are done sequentially. In another embodiment, the administration of the Hsp90 inhibitor and the VEGF inhibitor are dosed independently. In any one of these embodiments, the VEGF inhibitor may be bevacizumab, sunitinib, or sorafenib. In any one of these embodiments, the Hsp90 inhibitor may be a compound represented by formulae (I) or (Ia) or a compound in Tables 1 or 2.

In one embodiment, the method provides a kit for administration of the combination therapy having separate pharmaceutical compositions containing the Hsp90 inhibitor according to formulae (I) or (Ia), or a compound in Tables 1 or 2, and the VEGF inhibitor. In another embodiment, the kit includes one pharmaceutical composition containing both the Hsp90 inhibitor and the VEGF inhibitor in the same composition. In any of these embodiments, each pharmaceutical composition may include one or more pharmaceutically acceptable carrier or diluent. In any one of these embodiments, the VEGF inhibitor may be bevacizumab, sunitinib, or sorafenib. In any one of these embodiments, the Hsp90 inhibitor may be a compound represented in Tables 1 or 2.

In one embodiment, the method includes use of an Hsp90 inhibitor according to formulae (I) or (Ia) or a compound in Tables 1 or 2 for the manufacture of a medicament for treating cancer in combination with a VEGF inhibitor.

In certain embodiments, the treatments utilize an Hsp90 inhibitory compound according to formulae (I) or (Ia) or a compound in Tables 1 or 2 with a VEGF inhibitor to help to arrest, partially or fully, or reduce the development of multidrug resistant cancerous cells in a subject. In this embodiment, the combinations may allow a reduced efficacious amount of the VEGF inhibitor given to a subject, because the Hsp90 inhibitor should inhibit the development of multidrug-resistant cancerous cells. In one embodiment, the VEGF inhibitor may be bevacizumab, sunitinib, or sorafenib. In another embodiment, the VEGF inhibitor is bevacizumab.

### BRIEF DESCRIPTION OF THE DRAWINGS

The foregoing and other objects, features and advantages of the invention will be apparent from the following more particular description of some embodiments of the invention, as illustrated in the accompanying drawings in which like reference characters refer to the same parts throughout the different views. The drawings are not necessarily to scale, emphasis instead being placed upon illustrating the principles of the invention.

FIG. 1 displays the results of a nude mouse xenograft study on the effect of Compound 26 on the in vivo growth rate of RERF-LC-AI^{IVP} human lung tumor cells. Tumor bearing animals (8 mice/group) were i.p. injected 5 times per week for a total of 15 doses (hatched bar) and the average tumor volumes for each group (error bars represent SEM) were determined every 3-4 days. Treatment with a dose of 200 mg/kg body weight of Compound 26 inhibited tumor growth, as did a dose of 75 mg/kg body weight of 17-AAG (both compounds were dosed at approximately their maximum tolerated doses in nude mice).

FIG. 2 demonstrates that treatment with Compound 26 did not cause overt toxicity in a nude mouse xenograft model using RERF-LC-AI^{IVP} human lung tumor cells. Tumor bearing animals (8 mice/group) were i.p. injected 5 times per week for a total of 15 doses (hatched bar) and the cumulative average percent changes in body weights for each group relative to the start of dosing were determined every 2-3 days. Treatment with a dose of 200 mg/kg body weight of Compound 26 was not overtly toxic, as indicated by the minimal effects on the animal body weights in the test article-treated versus vehicle-treated groups.

FIG. 3 shows a SCID mouse xenograft study on the effects of the combination of Compound 1 plus bevacizumab on the in vivo growth rate of the human NSCLC cell line NCI-H1975. Tumor-bearing animals (8 mice/group) were injected 1 time per week for a total of 3 doses (arrowheads) with vehicle alone, Compound 1 alone, bevacizumab alone or a combination of Compound 1 and bevacizumab dosed concurrently. Compound 1 was i.v. injected and bevacizumab was i.p. injected. The average tumor volumes for each group (error bars represent SEM) were determined every 2-4 days. Single-agent treatments with either 50 mg/kg body weight of Compound 1, or 2 mg/kg body weight bevacizumab, moderately inhibited tumor growth. Concurrent treatment with a combination of 50 mg/kg body weight of Compound 1 plus 2 mg/kg body weight bevacizumab dramatically inhibited tumor growth and induced tumor regression. The efficacy observed for the combination treatment group was significantly greater than for either single agent alone (P<0.05; one-way ANOVA).

FIG. 4 shows another SCID mouse xenograft study on the effects of the combination of Compound 1 plus bevacizumab on the in vivo growth rate of the human NSCLC cell line NCI-H1975. Tumor-bearing animals (8 mice/group) were injected 1 time per week for a total of 4 doses (arrowheads) with vehicle alone, Compound 1 alone, bevacizumab alone or a combination of Compound 1 and bevacizumab dosed concurrently. Compound 1 was i.v. injected and bevacizumab was i.p. injected. The average tumor volumes for each group (error bars represent SEM) were determined every 2-3 days. Single-agent treatments with either 150 mg/kg body weight of Compound 1, or 10 mg/kg body weight bevacizumab, substantially inhibited tumor growth and moderately inhibited tumor growth, respectively. Concurrent treatment with a combination of 150 mg/kg body weight of Compound 1 plus 10 mg/kg body weight bevacizumab dramatically inhibited tumor growth and induced tumor regression. The efficacy observed for the combination treatment group was significantly greater than for either single agent alone (P<0.05; one-way ANOVA).

FIG. 5 shows a SCID mouse xenograft study on the effects of the combination of Compound 1 plus bevacizumab on the in vivo growth rate of the human multiple myeloma cell line RPMI 8226. Tumor-bearing animals (8 mice/group) were injected 5 time per week for a total of 15 doses (closed arrowheads) with vehicle or Compound 1 alone, and/or 2 times a week for a total 6 doses (open arrowheads) with vehicle or bevacizumab alone, or a combination of Compound 1 and bevacizumab dosed on these schedules. Compound 1 was i.v. injected and bevacizumab was i.p. injected. The average tumor volumes for each group (error bars represent SEM) were determined every 1-4 days. Single-agent treatments with either 12.5 mg/kg body weight of Compound 1, or 0.3 mg/kg body weight bevacizumab, moderately inhibited tumor growth. Concurrent treatment with a combination of 12.5 mg/kg body weight of Compound 1 plus 0.3 mg/kg body weight bevacizumab dramatically inhibited tumor growth and induced slight tumor regression. The efficacy observed for the combination treatment group was significantly greater than for either single agent alone (P<0.05; one-way ANOVA).

FIG. 6 shows the weight change for the animals used in the experiment depicted in FIG. 3.

FIG. 7 shows the weight change for the animals used in the experiment depicted in FIG. 4.

FIG. 8 shows an SCID mouse xenograft study on the effects of the combination of Compound 1 plus bevacizumab on the in vivo growth rate of the human NSCLC cell line HCC827. Tumor-bearing animals (8 mice/group) were injected 1 time per week for a total of 4 doses (arrowheads) with vehicle alone, Compound 1 alone, bevacizumab alone or a combination of Compound 1 and bevacizumab dosed concurrently. Compound 1 was i.v. injected and bevacizumab was i.p. injected. The average tumor volumes for each group (error bars represent SEM) were determined every 2-4 days. Single-agent treatments with either 150 mg/kg body weight of Compound 1, or 10 mg/kg body weight bevacizumab, moderately inhibited tumor growth. Concurrent treatment with a combination of 150 mg/kg body weight of Compound 1 plus 10 mg/kg body weight bevacizumab dramatically inhibited tumor growth and induced tumor regression. The efficacy observed for the combination treatment group was significantly greater than for either single agent alone (P<0.05; one-way ANOVA).

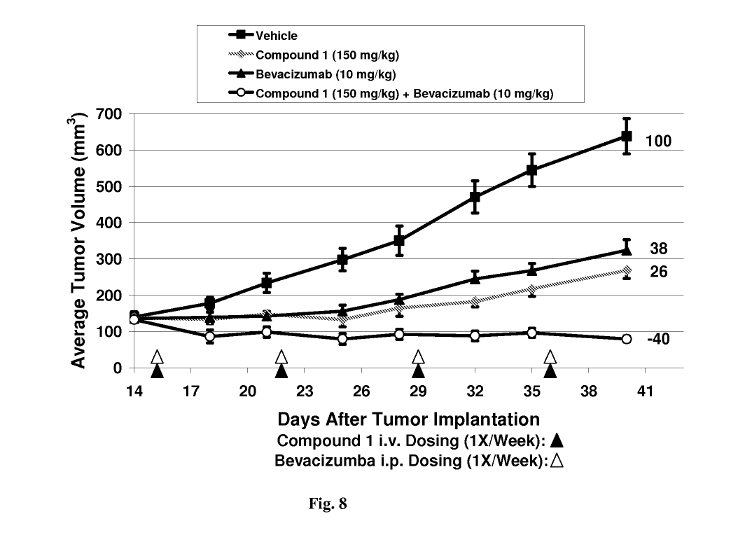

### DETAILED DESCRIPTION OF THE INVENTION

Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-l-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-l-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative halo alkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker Alkoxy groups included in compounds described herein may be optionally substituted with one or more substituents.

As used herein, a “haloalkoxy” is a haloalkyl group which is attached to another moiety via an oxygen linker

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C (NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹,—OC(O)OR³¹, —OC(S)OR³¹, —OC(NR³²)OR³¹, —SC(O)R³³, —SC(O)OR³¹, —SC(NR³²)OR³¹, —SC(S)R³³ —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C1-C4 alkyl, C1-C4 haloalkyl, C1-C4 alkoxy, C1-C4 haloalkoxy, C1-C4 hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

Unless indicated otherwise, the compounds described herein containing reactive functional groups, such as, for example, carboxy, hydroxy, thiol and amino moieties, also include corresponding protected derivatives thereof. “Protected derivatives” are those compounds in which a reactive site or sites are blocked with one ore more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

As used herein, the term “compound(s) described herein” or similar terms refers to a compound of formulae (I), or (Ia) or a compound in Tables 1 or 2 or a tautomer or pharmaceutically acceptable salt thereof. Also included in the scope of the embodiments are a solvate, clathrate, hydrate, polymorph, prodrug, or protected derivative of a compound of formulae (I), or (Ia), or a compound in Tables 1 or 2.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water).

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I) or (Ia) or a compound in Tables 1 or 2 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can typically be prepared using well-known methods, such as those described by BURGER'S MEDICINAL CHEMISTRY AND DRUG DISCOVERY, (Manfred E. Wolff Ed., 5^{th} ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

A “vascular endothelial growth factor inhibitor” or “VEGF inhibitor”, as used herein, includes any compound that disrupt the function of vascular endothelial growth factor A (VEGF) production within a cell. VEGF inhibitors are another important class of anticancer agents. These include monoclonal antibodies that bind VEGF to inactivate it, as well as VEGF receptor inhibitors. VEGF inhibitors include drugs such as bevacizumab (Avastin®), sunitinib (Sutent®), and sorafenib (Nexavar®). Humanized monoclonal antibodies such as bevacizumab that block VEGF disrupt the growth of blood vessels, angiogenesis, required for tumors to survive. Additionally, overproduction of VEGF can lead to non-cancerous vascular diseases such as age-related macular degeneration, wet macular degeneration, diabetic retinopathy, retinopathy of prematurity, myelofibrosis, diabetic macular edema, polypoidal choroidal vasculopathy or recurrent respiratory papillomatosis. Similar to monoclonal antibodies that block VEGF, VEGF receptor inhibitors function to block the growth of new blood vessels. Examples of VEGF receptor inhibitors include sunitinib and sorafenib. Monoclonal antibody therapies, such as bevacizumab, that block VEGF are described in U.S. Pat. Nos. 6,884,879, 7,060,269, and 7,297,334. Bevacizumab is an effective anticancer agent when used in combination with standard chemotherapies and is approved for use in metastatic colon cancer, metastatic non-small cell lung cancer, and metastatic breast cancer. It has the potential to be used in the treatment of colorectal cancer, non-metastatic breast cancer, non-small cell lung cancer, renal cell carcinoma, pancreatic cancer, ovarian cancer, hormone refractory prostate cancer, glioblastomas and liver cancer.

In addition to inhibiting angiogenesis, VEGF blockers and VEGF receptor inhibitors that block, occlude or otherwise disrupt neovascularization are useful in treating conditions such as choroidal neovascular membrane associated with age-related macular degeneration, neovascular retinopathy, diabetic macular edema, retinopathy of prematurity, macular edema secondary to retinal vein occlusions, pulmonary veno occlusive disease, central retinal vein occlusion, neovascular glaucoma, neoplastic meningitis, choroidal neovascularization, retinal neovascularization or corneal neovascularization.

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy, (e.g., diabetic retinopathy or other retinopathies), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis and desmoid tumors. Non-cancerous proliferative disorders also include hyperproliferation of cells in the skin such as psoriasis and its varied clinical forms, Reiter's syndrome, pityriasis rubra pilaris, hyperproliferative variants of disorders of keratinization (e.g., actinic keratosis, senile keratosis), scleroderma, and the like. In one embodiment, the proliferative disorder is a myeloproliferative disorder. In one aspect, the myeloproliferative disorder is polycythemia vera, idiopathic myelofirbrosis, myelodysplastic syndrome, psoriasis or essential thrombocythemia. In one embodiment, the proliferative disorder expresses JAK2V617F mutation of JAK2. In an aspect of this embodiment, the proliferative disorder is polycythemia vera, idiopathic myelofirbrosis, or essential thrombocythemia. In one aspect, the proliferative disorder is polycythemia vera.

As used herein, the term “pharmaceutically acceptable salt” refers to a salt prepared from a compound of formulae (I) or (Ia) or a compound in Tables 1 or 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I) or (Ia) or a compound in Tables 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I) or (Ia) or a compound in Tables 1 or 2. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound described herein are provided herein below. In a specific embodiment, the invention provides a method of treating, managing, or ameliorating a disease or disorder, e.g. a proliferative disorder, or one or more symptoms thereof, the method comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

The dosage of an individual VEGF inhibitor used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a proliferative disorder. In another embodiment, the proliferative disorder is cancer. The recommended dosages of therapeutic agents currently used for the treatment, management, or amelioration of a disease or disorder, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY:Mc-Graw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the VEGF inhibitor are dosed on independent schedules. In another embodiment, the Hsp90 inhibitor and the VEGF inhibitor are dosed on approximately the same schedule. In another embodiment, the Hsp90 inhibitor and the VEGF inhibitor are dosed concurrently or sequentially on the same day.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a “racemic mixture” means about 50% of one enantiomer and about 50% of is corresponding enantiomer of the molecule. The combination encompasses all enantiomerically-pure, enantiomerically-enriched, diastereomerically pure, diastereomerically enriched, and racemic mixtures of the compounds described herein. Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

The methods described herein utilize triazolone compounds listed in Tables 1 or 2, or a compound represented by Formulae (I) or (Ia):

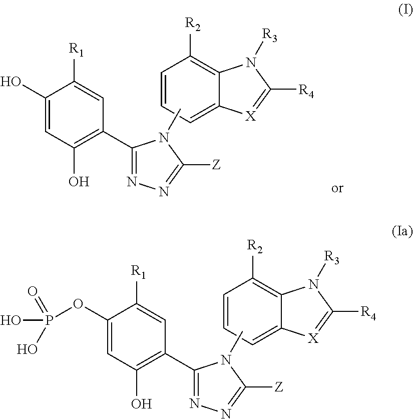

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NRONR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ is selected from the group consisting of —H, lower alkyl, lower alkoxy, lower cycloalkyl, and lower cycloalkoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy.

In another embodiment, in formula (I) or (Ia), R₃ is selected from the group consisting of —H, a lower alkyl, a lower cycloalkyl, —C(O)N(R₂₇)₂, and —C(O)OH, wherein R₂₇ is —H or a lower alkyl.

In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, R₄ is H or a lower alkyl.

In another embodiment, in formula (I) or (Ia), R₄ is selected from the group consisting of —H, methyl, ethyl, propyl, isopropyl or cyclopropyl.

In another embodiment, in formula (I) or (Ia), R₁ is selected from the group consisting of —H, —OH, —SH, —NH₂, a lower alkoxy and a lower alkyl amino.

In another embodiment, in formula (I) or (Ia), R₁ is selected from the group consisting of —H, —OH, methoxy and ethoxy.

In another embodiment, in formula (I) or (Ia), Z is —OH.

In another embodiment, in formula (I) or (Ia), Z is —SH.

In another embodiment, in formula (I) or (Ia), R₂ is selected from the group consisting of —H, —OH, —SH, —NH₂, a lower alkoxy and a lower alkyl amino.

In another embodiment, in formula (I) or (Ia), R₂ is selected from the group consisting of —H, —OH, methoxy, and ethoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH.

In another embodiment, the compound is selected from the group consisting of:

3-(2,4-dihydroxy-5-ethyl-phenyl)-4-(1,3-dimethyl-indol-5-yl)-5-hydroxy-[1,2,4]triazole,

3-(2,4-dihydroxy-5-isopropyl-phenyl)-4-(1,3-dimethyl-indol-5-yl)-5-hydroxy-[1,2,4]triazole,

3-(2,4-dihydroxy-5-isopropyl-phenyl)-4-(1-methyl-indol-5-yl)-5-hydroxy-[1,2,4]triazole,

3-(2,4-dihydroxy-5-isopropyl-phenyl)-4-(1-isopropyl-indol-4-yl)-5-hydroxy-[1,2,4]triazole,

3-(2,4-dihydroxy-5-isopropyl-phenyl)-4-(1-methyl-indazol-5-yl)-5-mercapto-[1,2,4]triazole,

3-(2,4-dihydroxy-5-isopropyl-phenyl)-4-(1-methyl-indazol-6-yl)-5-mercapto-[1,2,4]triazole,

3-(2,4-dihydroxyphenyl)-4-(1-ethyl-indol-4-yl)-5-mercapto-[1,2,4]triazole,

3-(2,4-dihydroxyphenyl)-4-(1-isopropyl-indol-4-yl)-5-mercapto-[1,2,4]triazole,

3-(2,4-dihydroxyphenyl)-4-(indol-4-yl)-5-mercapto-[1,2,4]triazole,

3-(2,4-dihydroxyphenyl)-4-(1-methoxyethyl-indol-4-yl)-5-mercapto-[1,2,4]triazole,

3-(2,4-dihydroxy-5-ethyl-phenyl)-4-(1-isopropyl-indol-4-yl)-5-mercapto-[1,2,4]triazole,

3-(2,4-dihydroxyphenyl)-4-(1-dimethylcarbamoyl-indol-4-yl)-5-mercapto-[1,2,4]triazole,

3-(2,4-dihydroxy-5-ethyl-phenyl)-4-(1-propyl-indol-4-yl)-5-mercapto-[1,2,4]triazole,

3-(2,4-dihydroxy-5-ethyl-phenyl)-4-(1,2,3-trimethyl-indol-5-yl)-5-mercapto-[1,2,4]triazole,

3-(2,4-dihydroxy-5-ethyl-phenyl)-4-(2,3-dimethyl-indol-5-yl)-5-mercapto-[1,2,4]triazole,

3-(2,4-dihydroxy-5-ethyl-phenyl)-4-(1-acetyl-2,3-dimethyl-indol-5-yl)-5-mercapto-[1,2,4]triazole,

3-(2,4-dihydroxy-5-ethyl-phenyl)-4-(1-propyl-2,3-dimethyl-indol-5-yl)-5-mercapto-[1,2,4]triazole,

3-(2,4-dihydroxy-5-ethyl-phenyl)-4-(1-n-butyl-indol-4-yl)-5-mercapto-[1,2,4]triazole,

3-(2,4-dihydroxy-5-ethyl-phenyl)-4-(1-n-pentyl-indol-4-yl)-5-mercapto-[1,2,4]triazole,

3-(2,4-dihydroxy-5-ethyl-phenyl)-4-(1-n-hexyl-indol-4-yl)-5-mercapto-[1,2,4]triazole, 3-(2,4-dihydroxy-5-cyclopropyl-phenyl)-4-(1-(1-methylcyclopropyl)-indol-4-yl)-5-mercapto-[1,2,4]triazole,

3-(2,4-dihydroxy-5-cyclopropyl-phenyl)-4-(1,2,3-trimethyl-indol-5-yl)-5-mercapto-[1,2,4]triazole,

3-(2,4-dihydroxy-5-ethyl-phenyl)-4-(1-methyl-3-ethyl-indol-5-yl)-5-mercapto-[1,2,4]triazole,

3-(2,4-dihydroxy-5-ethyl-phenyl)-4-(1,3-dimethyl-indol-5-yl)-5-mercapto-[1,2,4]triazole,

3-(2,4-dihydroxy-5-ethyl-phenyl)-4-(1-methyl-3-isopropyl-indol-5-yl)-5-mercapto-[1,2,4]triazole,

3-(2,4-dihydroxy-5-ethyl-phenyl)-4-(1,2-dimethyl-indol-5-yl)-5-mercapto-[1,2,4]triazole,

3-(2,4-dihydroxy-5-ethyl-phenyl)-4-(N-methyl-indol-5-yl)-5-mercapto-[1,2,4]triazole,

3-(2,4-dihydroxy-5-isopropyl-phenyl)-4-(1,3-dimethyl-indol-5-yl)-5-mercapto-[1,2,4]triazole,

3-(2,4-dihydroxy-5-cyclopropyl-phenyl)-4-(1,3-dimethyl-indol-5-yl)-5-mercapto-[1,2,4]triazole,

3-(2,4-dihydroxy-5-cyclopropyl-phenyl)-4-(1-methyl-indol-5-yl)-5-mercapto-[1,2,4]triazole,

3-(2,4-dihydroxy-5-isopropyl-phenyl)-4-(1H-indol-5-yl)-5-mercapto-[1,2,4]triazole,

3-(2,4-dihydroxy-5-ethyl-phenyl)-4-(1,2-dimethyl-indol-5-yl)-5-mercapto-[1,2,4]triazole,

3-(2,4-dihydroxy-5-isopropyl-phenyl)-4-(1-ethyl-indol-5-yl)-5-mercapto-[1,2,4]triazole,

3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, the compound is selected from the group consisting of

3-(2,4-dihydroxy-5-ethyl-phenyl)-4-(1-ethyl-benzimidazol-4-yl)-5-mercapto-[1,2,4]triazole,

3-(2,4-dihydroxy-5-ethyl-phenyl)-4-(1-ethyl-benzimidazol-4-yl)-5-mercapto-[1,2,4]triazole HCL salt,

3-(2,4-dihydroxy-5-ethyl-phenyl)-4-(2-methyl-3-ethyl-benzimidazol-5-yl)-5-mercapto-[1,2,4]triazole,

3-(2,4-dihydroxy-5-ethyl-phenyl)-4-(1-ethyl-2-methyl-benzimidazol-5-yl)-5-mercapto-[1,2,4]triazole,

3-(2,4-dihydroxy-5-isopropyl-pheny0-4-(1-methyl-2-trifluoromethyl-benzimidazol-5-yl)-5-mercapto-[1,2,4]triazole, or a tautomer, or a pharmaceutically acceptable salt thereof.

In another embodiment, the compound is selected from the group consisting of
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate, or a tautomer, or a pharmaceutically acceptable salt thereof.
   Hsp90 inhibitory compounds, as well as tautomers or pharmaceutically acceptable salts thereof, that may be used in the methods described herein are depicted in Tables 1 or 2.

TABLE 1

| | STRUCTURE | TAUTOMERIC STRUCTURE | NAME |
|---|---|---|---|
| 1 | 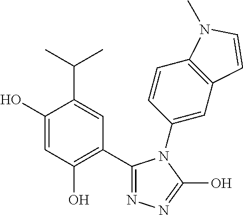 [SMILES: CC(C)c1cc(-c2nnc(O)n2-c2ccc3c(ccn3C)c2)c(O)cc1O] | 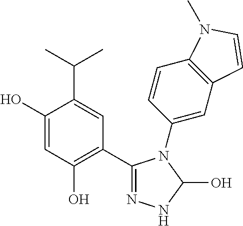 [SMILES: CC(C)c1cc(C2=NNC(O)N2c2ccc3c(ccn3C)c2)c(O)cc1O] | 3-(2,4-DIHYDROXY-5- ISOPROPYL-PHENYL)-4-(1- METHYL-INDOL-5-YL)-5- HYDROXY-[1,2,4] TRIAZOLE |
| 2 | 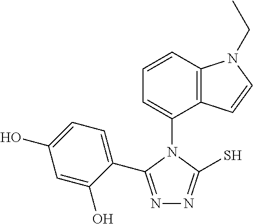 [SMILES: CCn1ccc2c(-n3c(S)nnc3-c3ccc(O)cc3O)cccc21] | 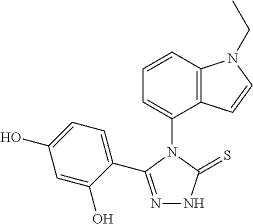 [SMILES: CCn1ccc2c(-n3c(-c4ccc(O)cc4O)n[nH]c3=S)cccc21] | 3-(2,4-DIHYDROXYPHENYL)-4- (1-ETHYL-INDOL-4-YL)-5- MERCAPTO-[1,2,4] TRIAZOLE |
| 3 | 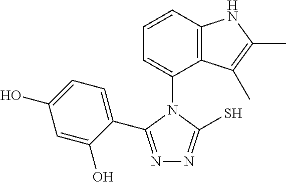 [SMILES: Cc1[nH]c2cccc(-n3c(S)nnc3-c3ccc(O)cc3O)c2c1C] | 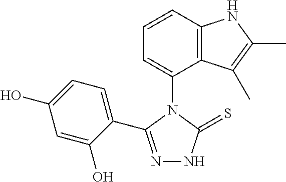 [SMILES: Cc1[nH]c2cccc(-n3c(-c4ccc(O)cc4O)n[nH]c3=S)c2c1C] | 3-(2,4-DIHYDROXY-PHENYL)-4- (2,3-DIMETHYL-1H-INDOL-4- YL)-5-MERCAPTO-[1,2,4] TRIAZOLE |
| 4 | 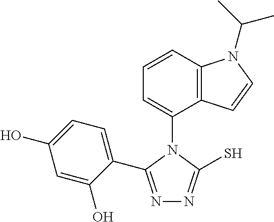 [SMILES: CC(C)n1ccc2c(-n3c(S)nnc3-c3ccc(O)cc3O)cccc21] | 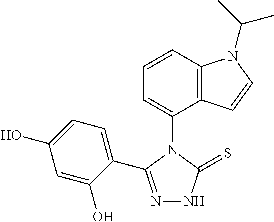 [SMILES: CC(C)n1ccc2c(-n3c(-c4ccc(O)cc4O)n[nH]c3=S)cccc21] | 3-(2,4-DIHYDROXYPHENYL)-4- (1-ISOPROPYL-INDOL-4-YL)-5- MERCAPTO-[1,2,4] TRIAZOLE |
| 5 | 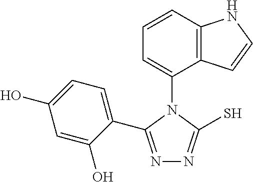 [SMILES: Oc1ccc(-c2nnc(S)n2-c2cccc3[nH]ccc23)c(O)c1] | 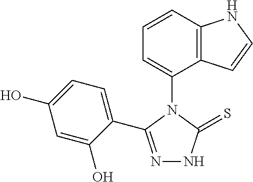 [SMILES: Oc1ccc(-c2n[nH]c(=S)n2-c2cccc3[nH]ccc23)c(O)c1] | 3-(2,4-DIHYDROXY-PHENYL)- 4-(INDOL-4-YL)-5-MERCAPTO- [1,2,4] TRIAZOLE |
| 6 | 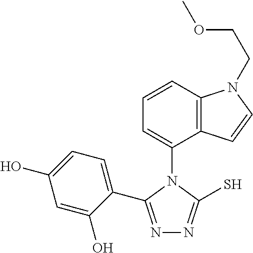 [SMILES: COCCn1ccc2c(-n3c(S)nnc3-c3ccc(O)cc3O)cccc21] | 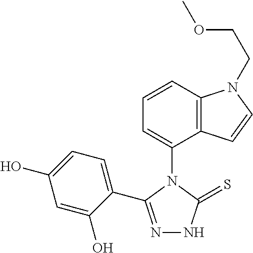 [SMILES: COCCn1ccc2c(-n3c(-c4ccc(O)cc4O)n[nH]c3=S)cccc21] | 3-(2,4-DIHYDROXY-PHENYL)- 4-[1-(2-METHOXYETHOXY)- INDOL-4-YL]-5-MERCAPTO- [1,2,4] TRIAZOLE |
| 7 | 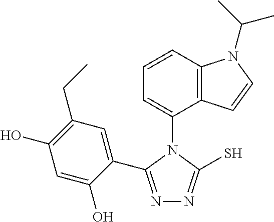 [SMILES: CCc1cc(-c2nnc(S)n2-c2cccc3c2ccn3C(C)C)c(O)cc1O] | 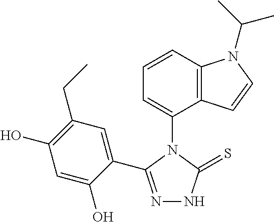 [SMILES: CCc1cc(-c2n[nH]c(=S)n2-c2cccc3c2ccn3C(C)C)c(O)cc1O] | 3-(2,4-DIHYDROXY-5-ETHYL- PHENYL)-4-(1-ISOPROPYL- INDOL-4-YL)-5-MERCAPTO- [1,2,4] TRIAZOLE |
| 8 | 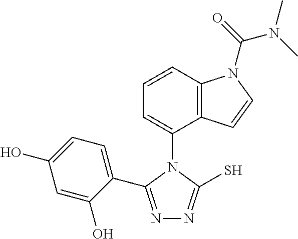 [SMILES: CN(C)C(=O)n1ccc2c(-n3c(S)nnc3-c3ccc(O)cc3O)cccc21] | 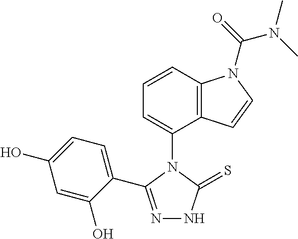 [SMILES: CN(C)C(=O)n1ccc2c(-n3c(-c4ccc(O)cc4O)n[nH]c3=S)cccc21] | 3-(2,4-DIHYDROXY-5-ETHYL- PHENYL)-4-[1-(DIMETHYL- CARBAMOYL)-INDOL-4-YL]-5- MERCAPTO-[1,2,4] TRIAZOLE |
| 9 | 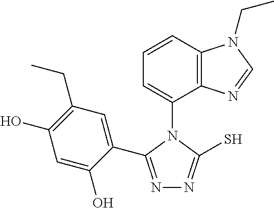 [SMILES: CCc1cc(-c2nnc(S)n2-c2cccc3c2ncn3CC)c(O)cc1O] | 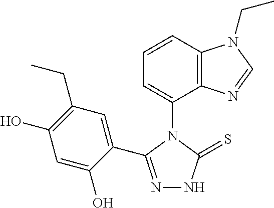 [SMILES: CCc1cc(-c2n[nH]c(=S)n2-c2cccc3c2ncn3CC)c(O)cc1O] | 3-(2,4-DIHYDROXY-5-ETHYL- PHENYL)-4-(1-ETHYL- BENZOIMIDAZOL-4-YL)-5- MERCAPTO-[1,2,4] TRIAZOLE |
| 10 | 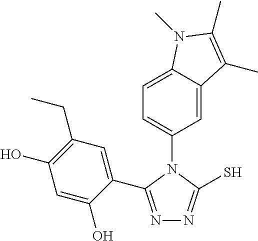 [SMILES: CCc1cc(-c2nnc(S)n2-c2ccc3c(c2)c(C)c(C)n3C)c(O)cc1O] | 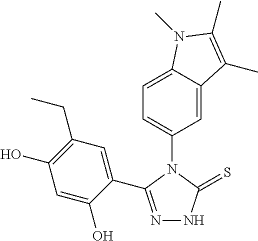 [SMILES: CCc1cc(-c2n[nH]c(=S)n2-c2ccc3c(c2)c(C)c(C)n3C)c(O)cc1O] | 3-(2,4-DIHYDROXY-5-ETHYL- PHENYL)-4-(1,2,3-TRIMETHYL- INDOL-5-YL)-5-MERCAPTO- [1,2,4] TRIAZOLE |
| 11 | 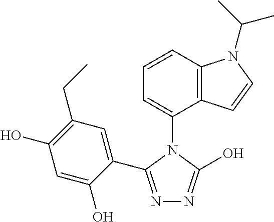 [SMILES: CCc1cc(-c2nnc(O)n2-c2cccc3c2ccn3C(C)C)c(O)cc1O] | 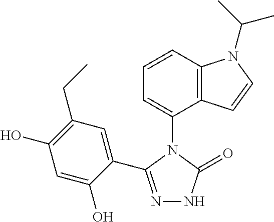 [SMILES: CCc1cc(-c2n[nH]c(=O)n2-c2cccc3c2ccn3C(C)C)c(O)cc1O] | 3-(2,4-DIHYDROXY-5-ETHYL- PHENYL)-4-(1-ISOPROPYL- INDOL-3-YL)-5-HYDROXY- [1,2,4] TRIAZOLE |
| 12 | 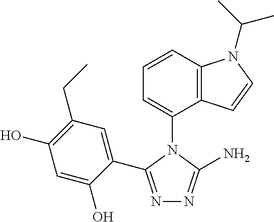 [SMILES: CCc1cc(-c2nnc(N)n2-c2cccc3c2ccn3C(C)C)c(O)cc1O] | 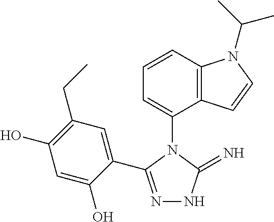 [SMILES: CCc1cc(-c2n[nH]c(=N)n2-c2cccc3c2ccn3C(C)C)c(O)cc1O] | 3-(2,4-DIHYDROXY-5-ETHYL- PHENYL)-4-(1-ISOPROPYL- INDOL-4-YL)-5-AMINO-[1,2,4] TRIAZOLE |
| 15 | 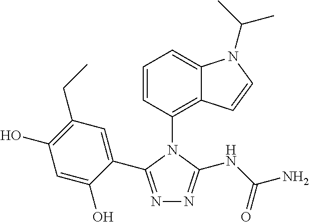 [SMILES: CCc1cc(-c2nnc(NC(N)=O)n2-c2cccc3c2ccn3C(C)C)c(O)cc1O] | | 3-(2,4-DIHYDROXY-5-ETHYL- PHENYL)-4-(1-ISOPROPYL- INDOL-4-YL)-5-UREIDO-[1,2,4] TRIAZOLE |
| 16 | 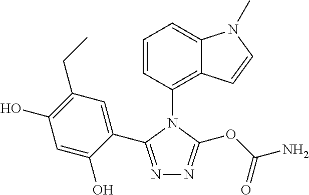 [SMILES: CCc1cc(-c2nnc(OC(N)=O)n2-c2cccc3c2ccn3C)c(O)cc1O] | | 3-(2,4-DIHYDROXY-5-ETHYL- PHENYL)-4-(1-METHYL- INDOL-4-YL)-5- CARBAMOYLOXY-[1,2,4] TRIAZOLE |
| 17 | 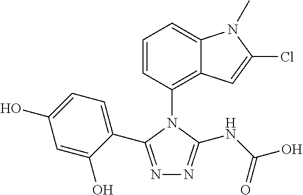 [SMILES: Cn1c(Cl)cc2c(-n3c(NC(=O)O)nnc3-c3ccc(O)cc3O)cccc21] | | 3-(2,4-DIHYDROXY-PHENYL)-4- (1-METHYL-2-CHLORO- INDOL-4-YL)-5- CARBAMOYLOXY-[1,2,4] TRIAZOLE |
| 18 | 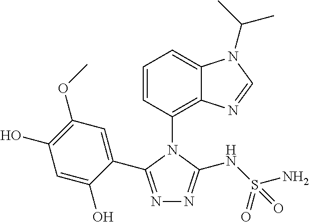 [SMILES: COc1cc(-c2nnc(NS(N)(=O)=O)n2-c2cccc3c2ncn3C(C)C)c(O)cc1O] | | 3-(2,4-DIHYDROXY- 5-METHOXY-PHENYL)-4- (1-ISOPROPYL- BENZOIMIDAZOL-4-YL)-5- (SULFAMOYLAMINO)-[1,2,4] TRIAZOLE |
| 20 | 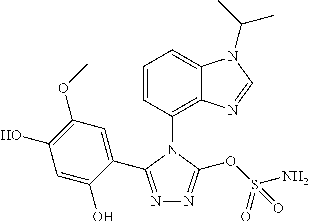 [SMILES: COc1cc(-c2nnc(OS(N)(=O)=O)n2-c2cccc3c2ncn3C(C)C)c(O)cc1O] | | 3-(2,4-HYDROXY-5- METHOXY-PHENYL)-4- (1-ISOPROPYL- BENZOIMIDAZOL-4-YL)-5- (SULFAMOYLOXY)-[1,2,4] TRIAZOLE |
| 21 | 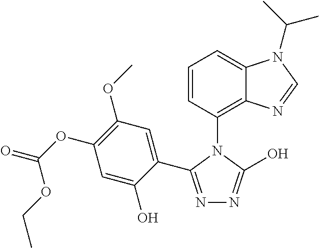 [SMILES: CCOC(=O)Oc1cc(O)c(-c2nnc(O)n2-c2cccc3c2ncn3C(C)C)cc1OC] | 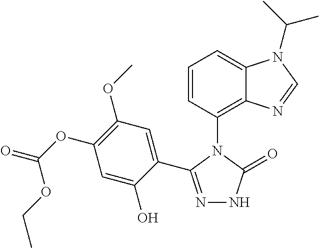 [SMILES: CCOC(=O)Oc1cc(O)c(-c2n[nH]c(=O)n2-c2cccc3c2ncn3C(C)C)cc1OC] | 3-(2-HYDROXY-4- ETHOXYCARBONYOXY-5- METHOXY-PHENYL)-4-(1- ISOPROPYL-BENZOIMIDAZOL- 4-YL)-5-HYDROXY-[1,2,4] TRIAZOLE |
| 22 | 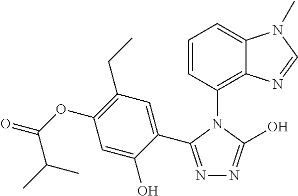 [SMILES: CCc1cc(-c2nnc(O)n2-c2cccc3c2ncn3C)c(O)cc1OC(=O)C(C)C] | 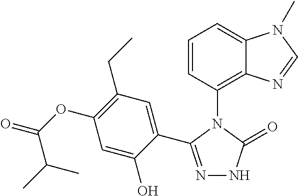 [SMILES: CCc1cc(-c2n[nH]c(=O)n2-c2cccc3c2ncn3C)c(O)cc1OC(=O)C(C)C] | 3-[2-HYDROXY-4- ISOBUTYRYLOXY-5-ETHYL- PHENYL]-4-(1-METHYL- BENZOIMIDAZOL-4-YL)-5- HYDROXY-[1,2,4] TRIAZOLE |
| 23 | 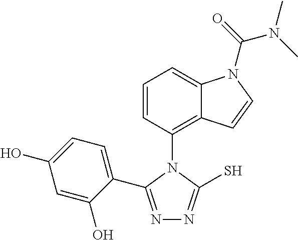 [SMILES: CN(C)C(=O)n1ccc2c(-n3c(S)nnc3-c3ccc(O)cc3O)cccc21] | 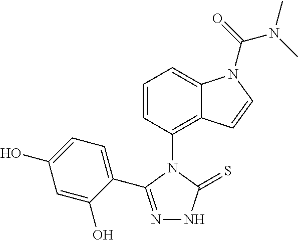 [SMILES: CN(C)C(=O)n1ccc2c(-n3c(-c4ccc(O)cc4O)n[nH]c3=S)cccc21] | 3-(2,4-DIHYDROXY-PHENYL)-4- (1-DIMETHYLCARBAMOYL- INDOL-4-YL)-5-MERCAPTO- [1,2,4] TRIAZOLE |
| 24 | 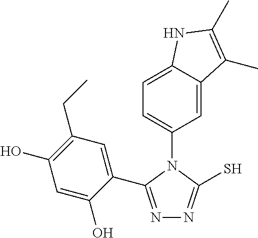 [SMILES: CCc1cc(-c2nnc(S)n2-c2ccc3[nH]c(C)c(C)c3c2)c(O)cc1O] | 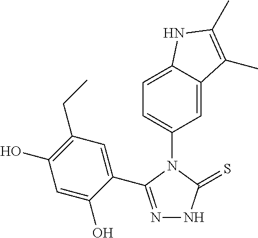 [SMILES: CCc1cc(-c2n[nH]c(=S)n2-c2ccc3[nH]c(C)c(C)c3c2)c(O)cc1O] | 3-(2,4-DIHYDROXY-5-ETHYL- PHENYL)-4-(2,3-DIMETHYL- INDOL-5-YL)-5-MERCAPTO- [1,2,4] TRIAZOLE |
| 25 | 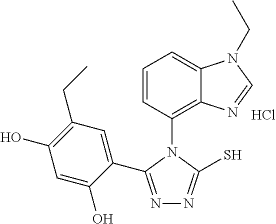 [SMILES: CCc1cc(-c2nnc(S)n2-c2cccc3c2ncn3CC)c(O)cc1O ; Cl] | 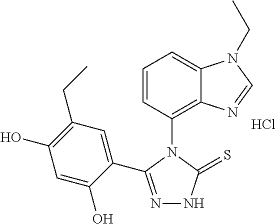 [SMILES: CCc1cc(-c2n[nH]c(=S)n2-c2cccc3c2ncn3CC)c(O)cc1O ; Cl] | 3-(2,4-DIHYDROXY-5-ETHYL- PHENYL)-4-(1-ETHYL-1H- BENZOIMIDAZOL-4-YL)-5- MERCAPTO-[1,2,4] TRIAZOLE, HCL SALT |
| 26 | 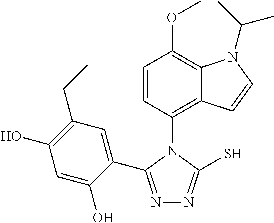 [SMILES: CCc1cc(-c2nnc(S)n2-c2ccc(OC)c3c2ccn3C(C)C)c(O)cc1O] | 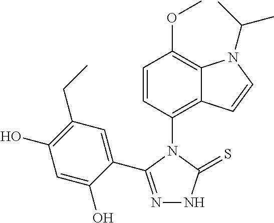 [SMILES: CCc1cc(-c2n[nH]c(=S)n2-c2ccc(OC)c3c2ccn3C(C)C)c(O)cc1O] | 3-(2,4-DIHYDROXY-5-ETHYL- PHENYL)-4-(1-ISOPROPYL-7- METHOXY-INDOL-4-YL)-5- MERCAPTO-[1,2,4] TRIAZOLE |
| 27 | 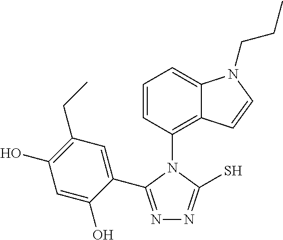 [SMILES: CCCn1ccc2c(-n3c(S)nnc3-c3cc(CC)c(O)cc3O)cccc21] | 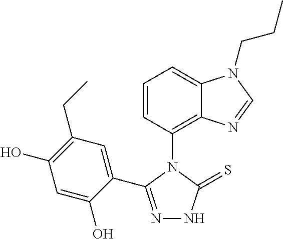 [SMILES: CCCn1cnc2c(-n3c(-c4cc(CC)c(O)cc4O)n[nH]c3=S)cccc21] | 3-(2,4-DIHYDROXY-5-ETHYL- PHENYL)-4-(1-PROPYL-INDOL- 4-YL)-5-MERCAPTO-[1,2,4] TRIAZOLE |
| 28 | 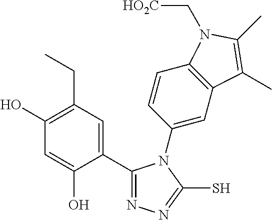 [SMILES: CCc1cc(-c2nnc(S)n2-c2ccc3c(c2)c(C)c(C)n3CC(=O)O)c(O)cc1O] | 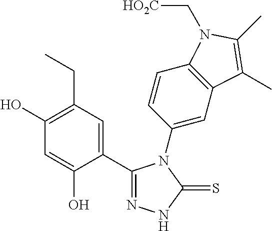 [SMILES: CCc1cc(-c2n[nH]c(=S)n2-c2ccc3c(c2)c(C)c(C)n3CC(=O)O)c(O)cc1O] | 3-(2,4-DIHYDROXY-5-ETHYL- PHENYL)-4-(1-ACETYL-2,3- DIMETHYL-INDOL-5-YL)-5- MERCAPTO-[1,2,4] TRIAZOLE |
| 29 | 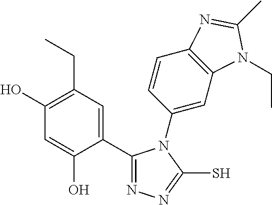 [SMILES: CCc1cc(-c2nnc(S)n2-c2ccc3nc(C)n(CC)c3c2)c(O)cc1O] | 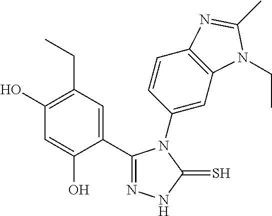 [SMILES: CCc1cc(-c2n[nH]c(=S)n2-c2ccc3nc(C)n(CC)c3c2)c(O)cc1O] | 3-(2,4-DIHYDROXY-5-ETHYL- PHENYL)-4-(2-METHYL-3- ETHYL-BENZIMIDAZOL- 5-YL)-5-MERCAPTO-[1,2,4] TRIAZOLE |
| 30 | 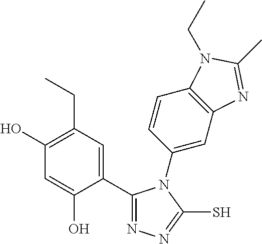 [SMILES: CCc1cc(-c2nnc(S)n2-c2ccc3c(c2)nc(C)n3CC)c(O)cc1O] | 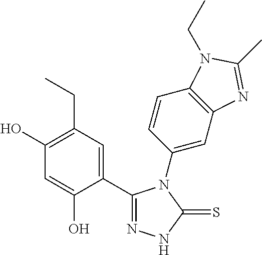 [SMILES: CCc1cc(-c2n[nH]c(=S)n2-c2ccc3c(c2)nc(C)n3CC)c(O)cc1O] | 3-(2,4-DIHYDROXY-5-ETHYL- PHENYL)-4-(1-ETHYL-2- METHYL-BENZIMIDAZOL- 5-YL)-5-MERCAPTO-[1,2,4] TRIAZOLE |
| 31 | 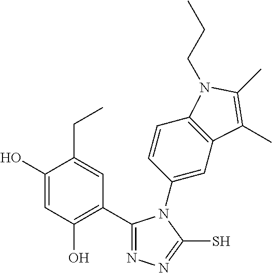 [SMILES: CCCn1c(C)c(C)c2cc(-n3c(S)nnc3-c3cc(CC)c(O)cc3O)ccc21] | 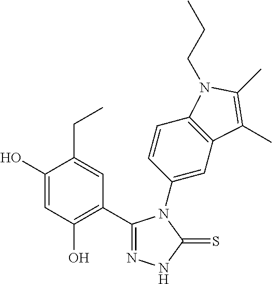 [SMILES: CCCn1c(C)c(C)c2cc(-n3c(-c4cc(CC)c(O)cc4O)n[nH]c3=S)ccc21] | 3-(2,4-DIHYDROXY-5-ETHYL- PHENYL)-4-(1-PROPYL-2,3- DIMETHYL-INDOL-5-YL)-5- MERCAPTO-[1,2,4] TRIAZOLE |
| 34 | 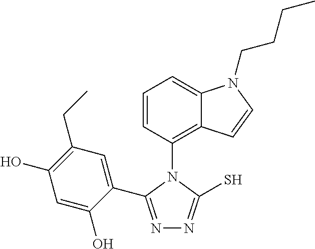 [SMILES: CCCCn1ccc2c(-n3c(S)nnc3-c3cc(CC)c(O)cc3O)cccc21] | 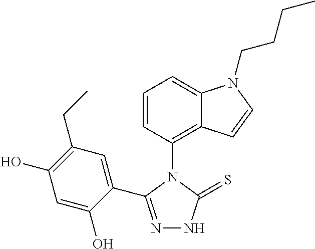 [SMILES: CCCCn1ccc2c(-n3c(-c4cc(CC)c(O)cc4O)n[nH]c3=S)cccc21] | 3-(2,4-DIHYDROXY-5-ETHYL- PHENYL)-4-(1-N-BUTYL- INDOL-4-YL)-5-MERCAPTO- [1,2,4] TRIAZOLE |
| 35 | 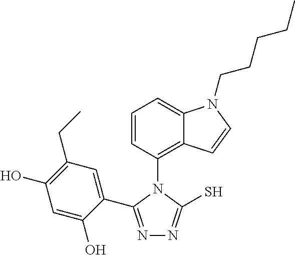 [SMILES: CCCCCn1ccc2c(-n3c(S)nnc3-c3cc(CC)c(O)cc3O)cccc21] | 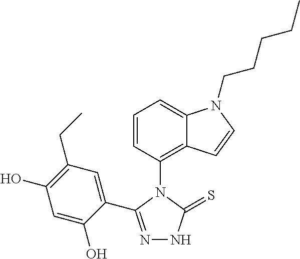 [SMILES: CCCCCn1ccc2c(-n3c(-c4cc(CC)c(O)cc4O)n[nH]c3=S)cccc21] | 3-(2,4-DIHYDROXY-5-ETHYL- PHENYL)-4-(1-N-PENTYL- INDOL-4-YL)-5-MERCAPTO- [1,2,4] TRIAZOLE |
| 36 | 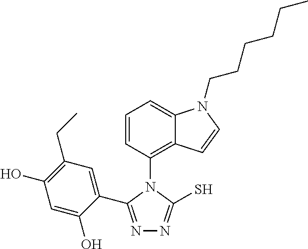 [SMILES: CCCCCCn1ccc2c(-n3c(S)nnc3-c3cc(CC)c(O)cc3O)cccc21] | 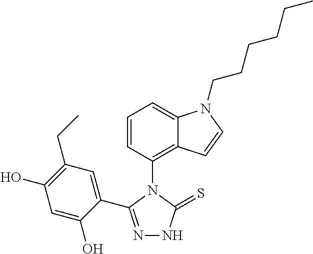 [SMILES: CCCCCCn1ccc2c(-n3c(-c4cc(CC)c(O)cc4O)n[nH]c3=S)cccc21] | 3-(2,4-DIHYDROXY-5-ETHYL- PHENYL)-4-(1-N-HEXYL- INDOL-4-YL)-5-MERCAPTO- [1,2,4] TRIAZOLE |
| 37 | 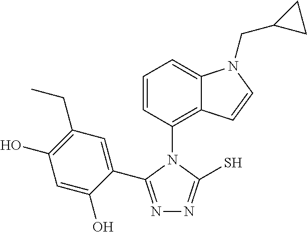 [SMILES: CCc1cc(-c2nnc(S)n2-c2cccc3c2ccn3CC2CC2)c(O)cc1O] | 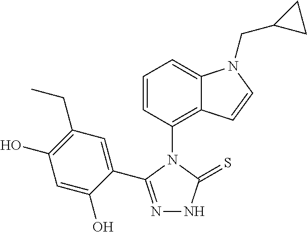 [SMILES: CCc1cc(-c2n[nH]c(=S)n2-c2cccc3c2ccn3CC2CC2)c(O)cc1O] | 3-(2,4-DIHYDROXY-5- CYCLOPROPYL-PHENYL)- 4-(1-(1-METHYL- CYCLOPROPYL)-INDOL- 4-YL)-5-MERCAPTO-[1,2,4] TRIAZOLE |
| 38 | 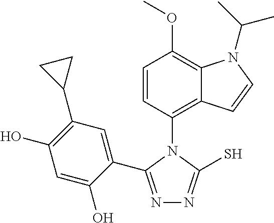 [SMILES: COc1ccc(-n2c(S)nnc2-c2cc(C3CC3)c(O)cc2O)c2ccn(C(C)C)c12] | 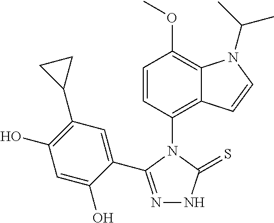 [SMILES: COc1ccc(-n2c(-c3cc(C4CC4)c(O)cc3O)n[nH]c2=S)c2ccn(C(C)C)c12] | 3-(2,4-DIHYDROXY-5- CYCLOPROPYL-PHENYL)-4-(1- ISOPROPYL-7-METHOXY- INDOL-4-YL)-5-MERCAPTO- [1,2,4] TRIAZOLE |
| 39 | 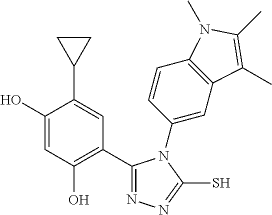 [SMILES: Cc1c(C)n(C)c2ccc(-n3c(S)nnc3-c3cc(C4CC4)c(O)cc3O)cc12] | 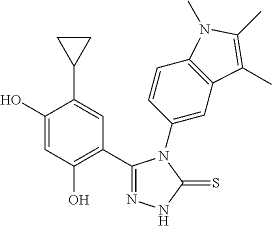 [SMILES: Cc1c(C)n(C)c2ccc(-n3c(-c4cc(C5CC5)c(O)cc4O)n[nH]c3=S)cc12] | 3-(2,4-DIHYDROXY-5- CYCLOPROPYL-PHENYL)-4- (1,2,3-TRIMETHYL- INDOL-5-YL)-5-MERCAPTO- [1,2,4] TRIAZOLE |
| 40 | 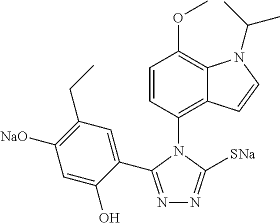 | | 3-(2,4-DIHYDROXY-5-ETHYL- PHENYL)-4-(1-ISOPROPYL-7- METHOXY-INDOL-4-YL)-5- MERCAPTO-[1,2,4] TRIAZOLE DISODIUM SALT |
| 41 | 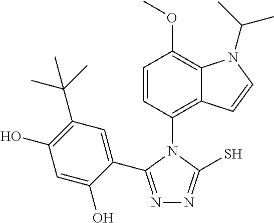 [SMILES: COc1ccc(-n2c(S)nnc2-c2cc(C(C)(C)C)c(O)cc2O)c2ccn(C(C)C)c12] | 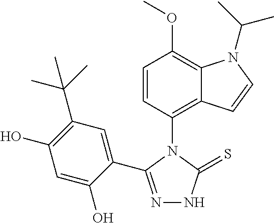 [SMILES: COc1ccc(-n2c(-c3cc(C(C)(C)C)c(O)cc3O)n[nH]c2=S)c2ccn(C(C)C)c12] | 3-(2,4-DIHDYROXY-5-TERT- BUTYL-PHENYL)-4-(1- ISOPROPYL-7-METHOXY- INDOL-4-YL)-5-MERCAPTO- [1,2,4] TRIAZOLE |
| 42 | 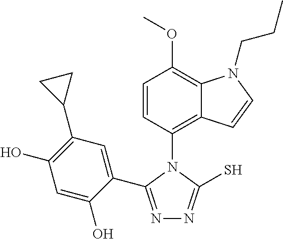 [SMILES: CCCn1ccc2c(-n3c(S)nnc3-c3cc(C4CC4)c(O)cc3O)ccc(OC)c21] | 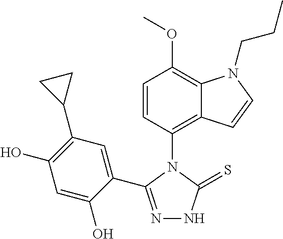 [SMILES: CCCn1ccc2c(-n3c(-c4cc(C5CC5)c(O)cc4O)n[nH]c3=S)ccc(OC)c21] | 3-(2,4-DIHYDROXY-5- CYCLOPROPYL-PHENYL)- 4-(1-PROPYL-7-METHOXY- INDOL-4-YL)-5- MERCAPTO-[1,2,4] TRIAZOLE |
| 43 | 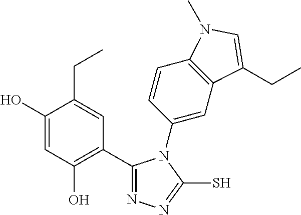 [SMILES: CCc1cc(-c2nnc(S)n2-c2ccc3c(c2)c(CC)cn3C)c(O)cc1O] | 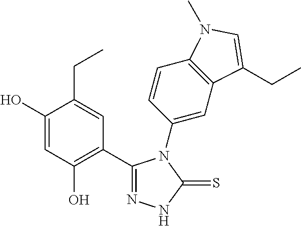 [SMILES: CCc1cc(-c2n[nH]c(=S)n2-c2ccc3c(c2)c(CC)cn3C)c(O)cc1O] | 3-(2,4-DIHYDROXY-5-ETHYL- PHENYL)-4-(1-METHYL-3- ETHYL-INDOL-5-YL)-5- MERCAPTO-[1,2,4] TRIAZOLE |
| 44 | 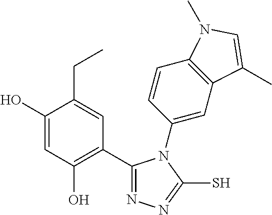 [SMILES: CCc1cc(-c2nnc(S)n2-c2ccc3c(c2)c(C)cn3C)c(O)cc1O] | 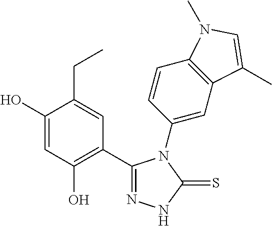 [SMILES: CCc1cc(-c2n[nH]c(=S)n2-c2ccc3c(c2)c(C)cn3C)c(O)cc1O] | 3-(2,4-DIHYDROXY-5-ETHYL- PHENYL)-4-(1,3-DIMETHYL- INDOL-5-YL)-5-MERCAPTO- [1,2,4] TRIAZOLE |
| 45 | 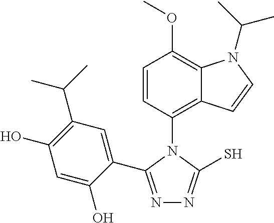 [SMILES: COc1ccc(-n2c(S)nnc2-c2cc(C(C)C)c(O)cc2O)c2ccn(C(C)C)c12] | 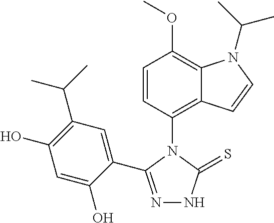 [SMILES: COc1ccc(-n2c(-c3cc(C(C)C)c(O)cc3O)n[nH]c2=S)c2ccn(C(C)C)c12] | 3-(2,4-DIHYDROXY-5- ISOPROPYL-PHENYL)-4-(1- ISOPROPYL-7-METHOXY- INDOL-4-YL)-5-MERCAPTO- [1,2,4] TRIAZOLE |
| 46 | 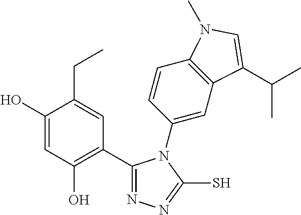 [SMILES: CCc1cc(-c2nnc(S)n2-c2ccc3c(c2)c(C(C)C)cn3C)c(O)cc1O] | 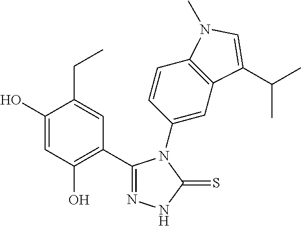 [SMILES: CCc1cc(-c2n[nH]c(=S)n2-c2ccc3c(c2)c(C(C)C)cn3C)c(O)cc1O] | 3-(2,4-DIHYDROXY-5-ETHYL- PHENYL)-4-(1-METHYL-3- ISOPROPYL-INDOL-5-YL)-5- MERCAPTO-[1,2,4] TRIAZOLE |
| 48 | 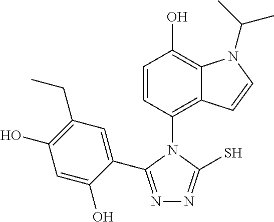 [SMILES: CCc1cc(-c2nnc(S)n2-c2ccc(O)c3c2ccn3C(C)C)c(O)cc1O] | 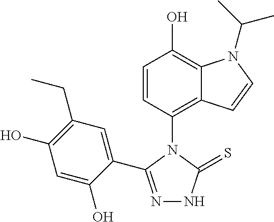 [SMILES: CCc1cc(-c2n[nH]c(=S)n2-c2ccc(O)c3c2ccn3C(C)C)c(O)cc1O] | 3-(2,4-DIHYDROXY-5-ETHYL- PHENYL)-4-(1-ISOPROPYL-7- HYDROXY-INDOL-4-YL)-5- MERCAPTO-[1,2,4] TRIAZOLE |
| 49 | 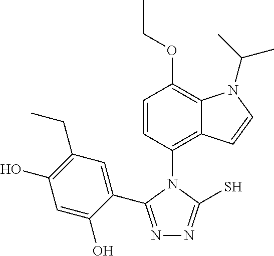 [SMILES: CCOc1ccc(-n2c(S)nnc2-c2cc(CC)c(O)cc2O)c2ccn(C(C)C)c12] | 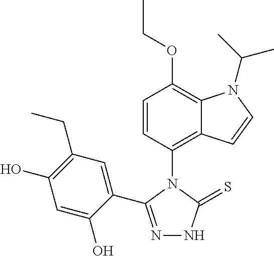 [SMILES: CCOc1ccc(-n2c(-c3cc(CC)c(O)cc3O)n[nH]c2=S)c2ccn(C(C)C)c12] | 3-(2,4-DIHYDROXY-5-ETHYL- PHENYL)-4-(1-ISOPROPYL-7- ETHOXY-INDOL-4-YL)-5- MERCAPTO-[1,2,4] TRIAZOLE |
| 50 | 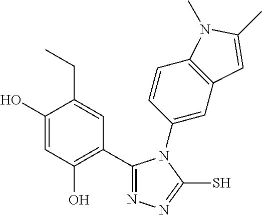 [SMILES: CCc1cc(-c2nnc(S)n2-c2ccc3c(c2)cc(C)n3C)c(O)cc1O] | 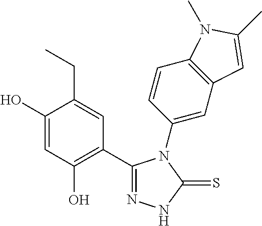 [SMILES: CCc1cc(-c2n[nH]c(=S)n2-c2ccc3c(c2)cc(C)n3C)c(O)cc1O] | 3-(2,4-DIHYDROXY-5-ETHYL- PHENYL)-4-(1,2-DIMETHYL- INDOL-5-YL)-5-MERCAPTO- [1,2,4] TRIAZOLE |
| 51 | 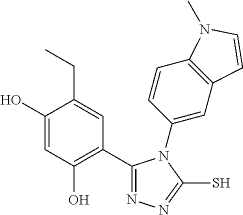 [SMILES: CCc1cc(-c2nnc(S)n2-c2ccc3c(ccn3C)c2)c(O)cc1O] | 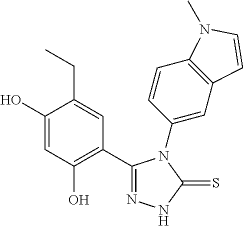 [SMILES: CCc1cc(-c2n[nH]c(=S)n2-c2ccc3c(ccn3C)c2)c(O)cc1O] | 3-(2,4-DIHYDROXY-5-ETHYL- PHENYL)-4-(N-METHYL- INDOL-5-YL)-5-MERCAPTO- [1,2,4] TRIAZOLE |
| 55 | 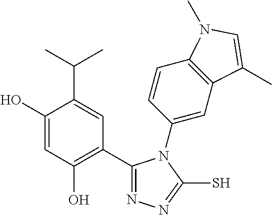 [SMILES: Cc1cn(C)c2ccc(-n3c(S)nnc3-c3cc(C(C)C)c(O)cc3O)cc12] | 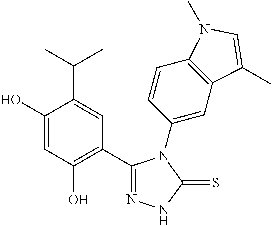 [SMILES: Cc1cn(C)c2ccc(-n3c(-c4cc(C(C)C)c(O)cc4O)n[nH]c3=S)cc12] | 3-(2,4-DIHYDROXY-5- ISOPROPYL-PHENYL)-4-(1,3- DIMETHYL-INDOL-5-YL)-5- MERCAPTO-[1,2,4] TRIAZOLE |
| 56 | 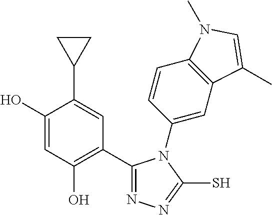 [SMILES: Cc1cn(C)c2ccc(-n3c(S)nnc3-c3cc(C4CC4)c(O)cc3O)cc12] | 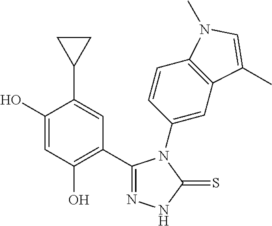 [SMILES: Cc1cn(C)c2ccc(-n3c(-c4cc(C5CC5)c(O)cc4O)n[nH]c3=S)cc12] | 3-(2,4-DIHYDROXY-5- CYCLOPROPYL-PHENYL)-4- (1,3-DIMETHYL-INDOL- 5-YL)-5-MERCAPTO- [1,2,4] TRIAZOLE |
| 57 | 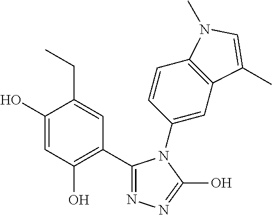 [SMILES: CCc1cc(-c2nnc(O)n2-c2ccc3c(c2)c(C)cn3C)c(O)cc1O] | 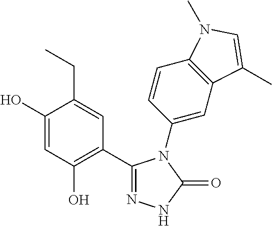 [SMILES: CCc1cc(-c2n[nH]c(=O)n2-c2ccc3c(c2)c(C)cn3C)c(O)cc1O] | 3-(2,4-DIHYDROXY-5-ETHYL- PHENYL)-4-(1,3-DIMETHYL- INDOL-5-YL)-5-HYDROXY- [1,2,4] TRIAZOLE |
| 58 | 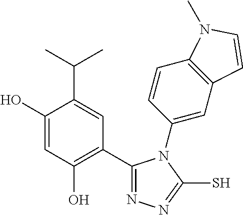 [SMILES: CC(C)c1cc(-c2nnc(S)n2-c2ccc3c(ccn3C)c2)c(O)cc1O] | 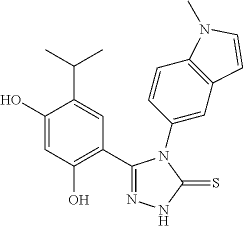 [SMILES: CC(C)c1cc(-c2n[nH]c(=S)n2-c2ccc3c(ccn3C)c2)c(O)cc1O] | 3-(2,4-DIHYDROXY-5- ISOPROPYL-PHENYL)-4-(N- METHYL-INDOL-5-YL)-5- MERCAPTO-[1,2,4] TRIAZOLE |
| 59 | 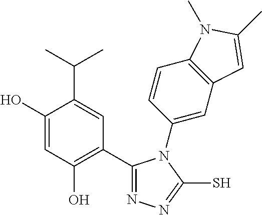 [SMILES: Cc1cc2cc(-n3c(S)nnc3-c3cc(C(C)C)c(O)cc3O)ccc2n1C] | 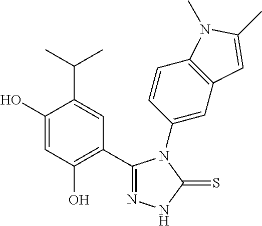 [SMILES: Cc1cc2cc(-n3c(-c4cc(C(C)C)c(O)cc4O)n[nH]c3=S)ccc2n1C] | 3-(2,4-DIHYDROXY-5- ISOPROPYL-PHENYL)-4-(1,2- DIMETHYL-INDOL-5-YL)-5- MERCAPTO-[1,2,4] TRIAZOLE |
| 60 | 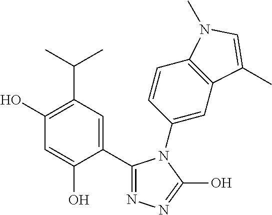 [SMILES: Cc1cn(C)c2ccc(-n3c(O)nnc3-c3cc(C(C)C)c(O)cc3O)cc12] | 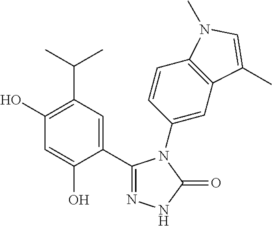 [SMILES: Cc1cn(C)c2ccc(-n3c(-c4cc(C(C)C)c(O)cc4O)n[nH]c3=O)cc12] | 3-(2,4-DIHYDROXY-5- ISOPROPYL-PHENYL)-4-(1,3- DIMETHYL-INDOL-5-YL)-5- HYDROXY-[1,2,4] TRIAZOLE |
| 62 | 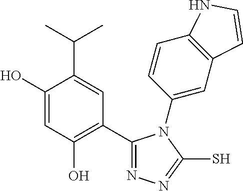 [SMILES: CC(C)c1cc(-c2nnc(S)n2-c2ccc3[nH]ccc3c2)c(O)cc1O] | 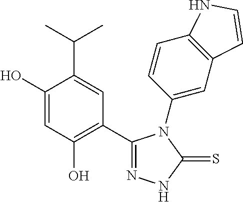 [SMILES: CC(C)c1cc(-c2n[nH]c(=S)n2-c2ccc3[nH]ccc3c2)c(O)cc1O] | 3-(2,4-DIHYDROXY-5- ISOPROPYL-PHENYL)-4-(1H- INDOL-5-YL)-5-MERCAPTO- [1,2,4] TRIAZOLE |
| 63 | 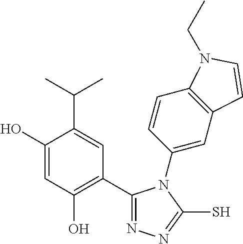 [SMILES: CCn1ccc2cc(-n3c(S)nnc3-c3cc(C(C)C)c(O)cc3O)ccc21] | 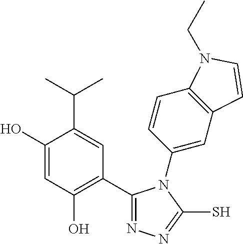 [SMILES: CCn1ccc2cc(-n3c(S)nnc3-c3cc(C(C)C)c(O)cc3O)ccc21] | 3-(2,4-DIHYDROXY-5- ISOPROPYL-PHENYL)-4-(1- ETHYL-INDOL-5-YL)-5- MERCAPTO-[1,2,4] TRIAZOLE |
| 64 | 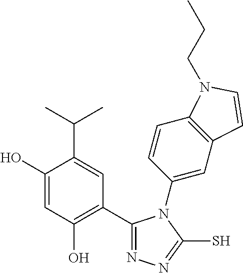 [SMILES: CCCn1ccc2cc(-n3c(S)nnc3-c3cc(C(C)C)c(O)cc3O)ccc21] | 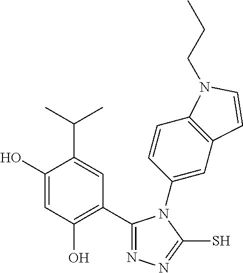 [SMILES: CCCn1ccc2cc(-n3c(S)nnc3-c3cc(C(C)C)c(O)cc3O)ccc21] | 3-(2,4-DIHYDROXY-5- ISOPROPYL-PHENYL)-4-(1- PROPYL-INDOL-5-YL)-5- MERCAPTO-[1,2,4] TRIAZOLE |
| 65 | 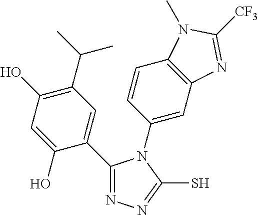 [SMILES: CC(C)c1cc(-c2nnc(S)n2-c2ccc3c(c2)nc(C(F)(F)F)n3C)c(O)cc1O] | 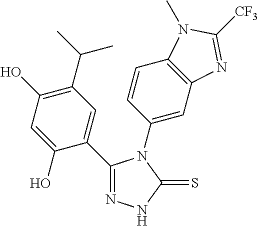 [SMILES: CC(C)c1cc(-c2n[nH]c(=S)n2-c2ccc3c(c2)nc(C(F)(F)F)n3C)c(O)cc1O] | 3-(2,4-DIHYDROXY-5- ISOPROPYL-PHENYL)-4-(1- METHYL-2- TRIFLUOROMETHYL- BENZIMIDAZOL-5-YL)-5- MERCAPTO-[1,2,4] TRIAZOLE |
| 66 | 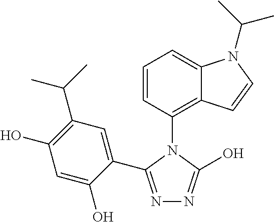 [SMILES: CC(C)c1cc(-c2nnc(O)n2-c2cccc3c2ccn3C(C)C)c(O)cc1O] | 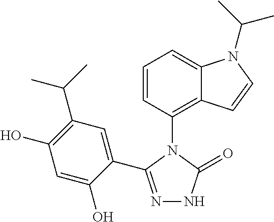 [SMILES: CC(C)c1cc(-c2n[nH]c(=O)n2-c2cccc3c2ccn3C(C)C)c(O)cc1O] | 3-(2,4-DIHYDROXY-5- ISOPROPYL-PHENYL)-4-(1- ISOPROPYL-INDOL-4-YL)-5- HYDROXY-[1,2,4] TRIAZOLE |

TABLE 2
Compounds according to Formula (Ia)

| NO. | STRUCTURE | TAUTOMERIC STRUCTURE | NAME |
|---|---|---|---|
| 1A | 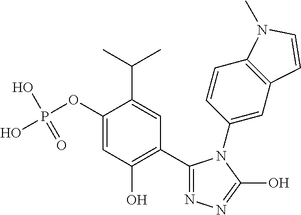 [SMILES: CC(C)c1cc(-c2nnc(O)n2-c2ccc3c(ccn3C)c2)c(O)cc1OP(=O)(O)O] | 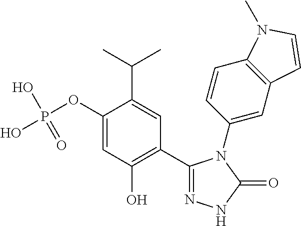 [SMILES: CC(C)c1cc(-c2n[nH]c(=O)n2-c2ccc3c(ccn3C)c2)c(O)cc1OP(=O)(O)O] | 5-HYDROXY-4-(5- HYDROXY-4-(1-METHYL- 1H-INDOL-5-YL)-4H- 1,2,4-TRIAZOL-3-YL)-2- ISOPROPYLPHENYL DIHYDROGEN PHOSPHATE |
| 2A | 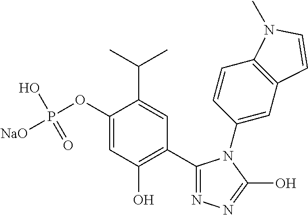 | 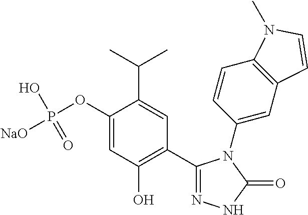 | SODIUM 5-HYDROXY-4- (5-HYDROXY-4-(1- METHYL-1H-INDOL-5- YL)-4H-1,2,4-TRIAZOL-3- YL)-2-ISOPROPYLPHENYL PHOSPHATE |
| 3A | 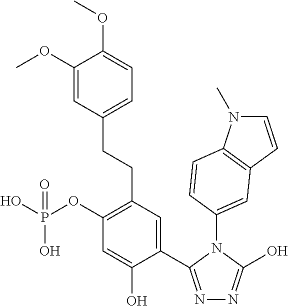 [SMILES: COc1ccc(CCc2cc(-c3nnc(O)n3-c3ccc4c(ccn4C)c3)c(O)cc2OP(=O)(O)O)cc1OC] | 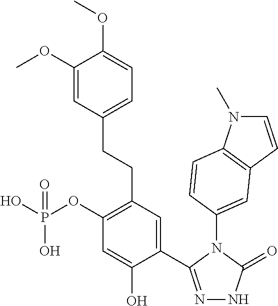 [SMILES: COc1ccc(CCc2cc(-c3n[nH]c(=O)n3-c3ccc4c(ccn4C)c3)c(O)cc2OP(=O)(O)O)cc1OC] | 2-(3,4- DIMETHOXYPHENETHYL)- 5-HYDROXY-4-(5- HYDROXY-4-(1-METHYL- 1H-INDOL-5-YL)-4H- 1,2,4-TRIAZOL-3- YL)PHENYL DIHYDROGEN PHOSPHATE |
| 4A | 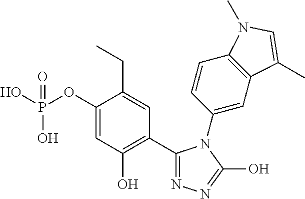 [SMILES: CCc1cc(-c2nnc(O)n2-c2ccc3c(c2)c(C)cn3C)c(O)cc1OP(=O)(O)O] | 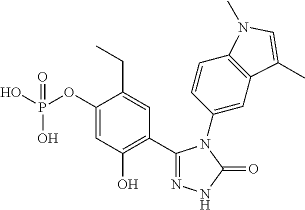 [SMILES: CCc1cc(-c2n[nH]c(=O)n2-c2ccc3c(c2)c(C)cn3C)c(O)cc1OP(=O)(O)O] | 4-(4-(1,3-DIMETHYL-1H- INDOL-5-YL)-5- HYDROXY-4H-1,2,4- TRIAZOL-3-YL)-2-ETHYL- 5-HYDROXYPHENYL DIHYDROGEN PHOSPHATE |

The Hsp90 inhibitory compounds used in the disclosed combination methods can be prepared according to the procedures disclosed in U.S. Patent Publication No. 2006/0167070, and WO2009/023211.

These triazolone compounds typically can form a tautomeric structure as shown below and as exemplified by the tautomeric structures shown in Tables 1 and 2:

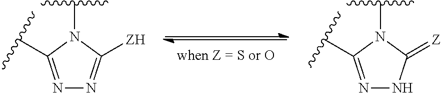

The present invention provides pharmaceutical combinations for the treatment, prophylaxis, and amelioration of proliferative disorders, such as cancer. In a specific embodiment, the combination comprises one or more Hsp90 inhibitors according to formulae (I) or (Ia), or a compound in Tables 1 or 2, or a tautomer or a pharmaceutically acceptable salt thereof in addition to a VEGF inhibitor.

In one embodiment, the combination includes a pharmaceutical composition or a single unit dosage form containing both an Hsp90 inhibitor and a VEGF inhibitor. Pharmaceutical combinations and dosage forms described herein comprise the two active ingredients in relative amounts and formulated in such a way that a given pharmaceutical combination or dosage form can be used to treat proliferative disorders, such as cancer. Preferred pharmaceutical combinations and dosage forms comprise a compound of formulae (I) or (Ia), or a compound in Tables 1 or 2, or a tautomer or pharmaceutically acceptable salt thereof, in combination with a VEGF inhibitor. In other embodiments, the Hsp90 inhibitor and the VEGF inhibitor may be in individual or separate pharmaceutical compositions, depending on the dosing schedules, preferred routes of administration, and available formulations of the two inhibitors. Optionally, these embodiments can also contain one or more additional therapeutic agents.

The pharmaceutical combinations described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the combination is formulated in accordance with routine procedures as a pharmaceutical composition adapted for intravenous, subcutaneous, intramuscular, oral, intranasal or topical administration to human beings. In one embodiment, the combination is formulated in accordance with routine procedures for subcutaneous administration to human beings.

In a specific embodiment, the combination therapies described herein comprise one or more compounds and at least one other therapy which has the same mechanism of action as the compounds. In another specific embodiment, the combination therapies described herein comprise one or more compounds described herein and at least one other therapy which has a different mechanism of action than the compounds. In certain embodiments, the combination therapies described herein improve the therapeutic effect of one or more triazolone compounds described herein by functioning together with the VEGF inhibitor to have an additive or synergistic effect. In certain embodiments, the combination therapies described herein reduce the side effects associated with the therapies. In certain embodiments, the combination therapies described herein reduce the effective dosage of one or more of the therapies.

In a specific embodiment, the combination comprising one or more triazolone compounds described herein is administered to a subject, preferably a human, to prevent, treat, manage, or ameliorate cancer, or one or more symptom thereof. In accordance with the invention, the pharmaceutical combinations described herein may also comprise one or more other agents being used, have been used, or are known to be useful in the treatment or amelioration of cancer, particularly metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma. The pharmaceutical combinations described herein utilize pharmaceutical compositions and dosage forms which comprise one or more excipients. Suitable excipients are well known to those skilled in the art of pharmacy.

The triazolone compounds described herein can be also formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

The present invention also provides a method of treating a proliferative disorder in a subject, comprising administering to the subject an effective amount of the combination of an Hsp90 inhibitor and a VEGF inhibitor as described herein. In one embodiment, the proliferative disorder is cancer. In one aspect of this embodiment, the cancer is metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma. In another aspect of this embodiment the cancer is metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, or non-metastatic unresectable liver cancer. In another aspect of this embodiment, the cancer is non-small cell lung cancer.

Smooth muscle cell proliferation includes hyperproliferation of cells in the vasculature, for example, intimal smooth muscle cell hyperplasia, restenosis and vascular occlusion, particularly stenosis following biologically- or mechanically-mediated vascular injury, e.g., vascular injury associated with angioplasty. Moreover, intimal smooth muscle cell hyperplasia can include hyperplasia in smooth muscle other than the vasculature, e.g., bile duct blockage, bronchial airways of the lung in patients with asthma, in the kidneys of patients with renal interstitial fibrosis, and the like.

In one embodiment, the disclosed method is believed to be effective in treating a subject with non-solid tumors such as multiple myeloma. In another embodiment, the disclosed method is believed to be effective against T-cell leukemia, e.g., as exemplified by Jurkat and CEM cell lines; B-cell leukemia, e.g., as exemplified by the SB cell line; promyelocytes, e.g., as exemplified by the HL-60 cell line; uterine sarcoma, e.g., as exemplified by the MES-SA cell line; monocytic leukemia, e.g., as exemplified by the THP-1(acute) cell line; and lymphoma, e.g., as exemplified by the U937 cell line.

Some of the disclosed methods can be also effective at treating subjects whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including gamma-radiation, neutron beam radiotherapy, electron beam radiotherapy, proton therapy, brachytherapy, and systemic radioactive isotopes), endocrine therapy, biologic response modifiers (including interferons, interleukins, and tumor necrosis factor (TNF)), hyperthermia and cryotherapy, agents to attenuate any adverse effects (e.g., antiemetics), and other approved chemotherapeutic drugs.

The therapeutic agents of the combination therapies described herein can be administered sequentially or concurrently. In one embodiment, the administration of the Hsp90 inhibitor and the VEGF inhibitor are done concurrently. In another embodiment, the administration of the Hsp90 inhibitor and the VEGF inhibitor are done separately. In another embodiment, the administration of the Hsp90 inhibitor and the VEGF inhibitor are done sequentially In one embodiment, the administration of the Hsp90 inhibitor and the VEGF inhibitor are done until the cancer is cured or stabilized or improved.

In one specific embodiment, the present method includes treating, managing, or ameliorating cancer, or one or more symptoms thereof, comprising administering to a subject in need thereof one or more compounds represented by the structural formulae (I) or (Ia) or a compound in Table 1 or Table 2, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of a VEGF inhibitor.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of bevacizumab.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of a VEGF inhibitor.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of bevacizumab.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In yet another embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a VEGF inhibitor such as bevacizumab, sunitinib, or sorafenib.

In one embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a VEGF inhibitor such as bevacizumab, sunitinib, or sorafenib.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with bevacizumab.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a VEGF inhibitor such as bevacizumab, sunitinib, or sorafenib.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with bevacizumab.

In one embodiment, the method of treating a subject with cancer, wherein the subject is being or has been treated with a chemotherapeutic agent, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In one embodiment, the method of treating a subject with cancer, wherein the subject is being or has been treated with a chemotherapeutic agent, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In one embodiment, the method of treating a subject with cancer, wherein the subject has proven refractory to other therapies but is no longer on these therapies, includes administering to the subject an effective amount of a triazolone compound represented by the structural formulae (I) or (Ia) or a compound in Table 1 or Table 2, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a VEGF inhibitor such as bevacizumab, sunitinib, or sorafenib.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with bevacizumab.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a VEGF inhibitor such as bevacizumab, sunitinib, or sorafenib.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with bevacizumab.

In one embodiment, the method of treating a subject with cancer, wherein the subject has proven refractory to other therapies but is no longer on these therapies, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In one embodiment, the method of treating a subject with cancer, wherein the subject has proven refractory to other therapies but is no longer on these therapies, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a VEGF inhibitor such as bevacizumab, sunitinib, or sorafenib, wherein the cancer is selected from the group consisting of metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

In one further embodiment, the method includes inhibiting the growth of a cancer or tumor cell comprising the steps of: (a) contacting the cell with an effective amount of a compound of formulae (I) or (Ia) or a compound in Table (1) or Table (2), or tautomer or a pharmaceutically acceptable salt thereof; and (b) exposing the cell to an effective amount of a VEGF inhibitor such as bevacizumab, sunitinib, or sorafenib.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of a VEGF inhibitor such as bevacizumab, sunitinib, or sorafenib.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of bevacizumab.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of a VEGF inhibitor such as bevacizumab, sunitinib, or sorafenib.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of bevacizumab.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, when the triazolone compounds described herein are administered in combination with a VEGF inhibitor, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies are administered within the same patient visit.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating a proliferative disorders, such as cancer, or one or more symptoms thereof, the methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

### EXAMPLES

### Example 1

### Compound 26 Displayed Anti-Tumor Activity against Human Tumor Cells in a nude Mouse Xenograft Model

The human squamous non-small cell lung cancer cell line, RERF-LC-AI (RCB0444; S. Kyoizumi, et al., Cancer. Res. 45:3274-3281, 1985), was obtained from the Riken Cell Bank (Tsukuba, Ibaraki, Japan). The cell line was cultured in growth media prepared from 50% Dulbecco's Modified Eagle Medium (high glucose), 50% RPMI Media 1640, 10% fetal bovine serum (FBS), 1% 100× L-glutamine, 1% 100× penicillin-streptomycin, 1% 100× sodium pyruvate and 1% 100× MEM non-essential amino acids. FBS was obtained from American Type Culture Collection (Manassas, Va., USA) and all other reagents were obtained from Invitrogen Corp. (Carlsbad, Calif., USA). Approximately 4-5×10(6) cells that had been cryopreserved in liquid nitrogen were rapidly thawed at 37° C. and transferred to a 175 cm² tissue culture flask containing 50 ml of growth media and then incubated at 37° C. in a 5% CO₂ incubator.

The growth media was replaced every 2-3 days until the flask became 90% confluent, typically in 5-7 days. To passage and expand the cell line, a 90% confluent flask was washed with 10 ml of room temperature phosphate buffered saline (PBS) and the cells were disassociated by adding 5 ml 1× trypsin-EDTA (Invitrogen) and incubating at 37° C. until the cells detached from the surface of the flask. To inactivate the trypsin, 5 ml of growth media was added and then the contents of the flask were centrifuged to pellet the cells. The supernatant was aspirated and the cell pellet was resuspended in 10 ml of growth media and the cell number determined using a hemocytometer. Approximately 1-3×10(6) cells per flask were seeded into 175 cm² flasks containing 50 ml of growth media and incubated at 37° C. in a 5% CO₂ incubator. When the flasks reached 90% confluence, the above passaging process was repeated until sufficient cells had been obtained for implantation into mice.

Seven to eight week old, female Crl:CD-1-nuBR (nude) mice were obtained from Charles River Laboratories (Wilmington, Mass., USA) Animals were housed 4-5/cage in micro-isolators, with a 12 hr/12 hr light/dark cycle, acclimated for at least 1 week prior to use and fed normal laboratory chow ad libitum. Studies were conducted on animals between 8 and 12 weeks of age at implantation. To implant RERF-LC-AI tumor cells into nude mice, the cells were trypsinized as above, washed in PBS and resuspended at a concentration of 50×10(6) cells/ml in 50% non-supplemented RPMI Media 1640 and 50% Matrigel Basement Membrane Matrix (#354234; BD Biosciences; Bedford, Mass., USA). Using a 27 gauge needle and 1 cc syringe, 0.1 ml of the cell suspension was injected subcutaneously into the flank of each nude mouse. Tumor volumes (V) were calculated by caliper measurement of the width (W), length (L) and thickness (T) of tumors using the following formula: V=0.5236×(L×W×T).

In vivo passaged RERF-LC-AI tumor cells (RERF-LC-AI^{IVP}) were isolated to improve the rate of tumor implantation relative to the parental cell line in nude mice. RERF-LC-AI tumors were permitted to develop in vivo until they reached approximately 250 mm³ in volume, which required approximately 3 weeks following implantation. Mice were euthanized via CO₂ asphyxiation and their exteriors sterilized with 70% ethanol in a laminar flow hood. Using sterile technique, tumors were excised and diced in 50 ml PBS using a scalpel blade. A single cell suspension was prepared using a 55 ml Wheaton Safe-Grind tissue grinder (catalog #62400-358; VWR International, West Chester, Pa., USA) by plunging the pestle up and down 4-5 times without twisting. The suspension was strained through a 70 μM nylon cell strainer and then centrifuged to pellet the cells. The resulting pellet was resuspended in 0.1 M NH₄Cl to lyse contaminating red blood cells and then immediately centrifuged to pellet the cells. The cell pellet was resuspended in growth media and seeded into 175 cm² flasks containing 50 ml of growth media at 1-3 tumors/flask or approximately 10×10(6) cells/flask. After overnight incubation at 37° C. in a 5% CO₂ incubator, non-adherent cells were removed by rinsing two times with PBS and then the cultures were fed with fresh growth media. When the flasks reached 90% confluence, the above passaging process was repeated until sufficient cells had been obtained for implantation into mice.

RERF-LC-A^{IVP} cells were then implanted as above and tumors were permitted to develop in vivo until the majority reached an average of 100-200 mm³ in tumor volume, which typically required 2-3 weeks following implantation Animals with oblong or very small or large tumors were discarded, and only animals carrying tumors that displayed consistent growth rates were selected for studies. Animals were randomized into treatment groups so that the average tumor volumes of each group were similar at the start of dosing.

The Hsp90 inhibitor, 17-allylamino-17-demethoxygeldanamycin (17-AAG), was employed as a positive control (Albany Molecular Research, Albany, N.Y., USA). Stock solutions of test articles were prepared by dissolving the appropriate amounts of each compound in dimethyl sulfoxide (DMSO) by sonication in an ultrasonic water bath. Stock solutions were prepared weekly, stored at −20° C. and diluted fresh each day for dosing. A solution of 20% Cremophore RH40 (polyoxyl 40 hydrogenated castor oil; BASF Corp., Aktiengesellschaft, Ludwigshafen, Germany) in 80% D5W (5% dextrose in water; Abbott Laboratories, North Chicago, Ill., USA) was also prepared by first heating 100% Cremophore RH40 at 50-60° C. until liquefied and clear, diluting 1:5 with 100% D5W, reheating again until clear and then mixing well. This solution was stored at room temperature for up to 3 months prior to use. To prepare formulations for daily dosing, DMSO stock solutions were diluted 1:10 with 20% Cremophore RH40. The final formulation for dosing contained 10% DMSO, 18% Cremophore RH40, 3.6% dextrose, 68.4% water and the appropriate amount of test article. Animals were intraperitoneally (i.p.) injected with this solution at 10 ml per kg body weight on a schedule of 5 days per week (Monday, Tuesday, Wednesday, Thursday and Friday, with no dosing on Saturday and Sunday) for a total of 15 doses.

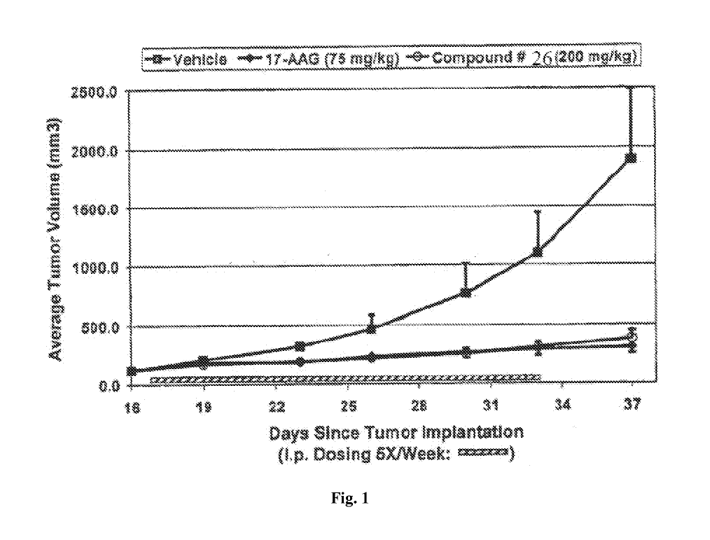

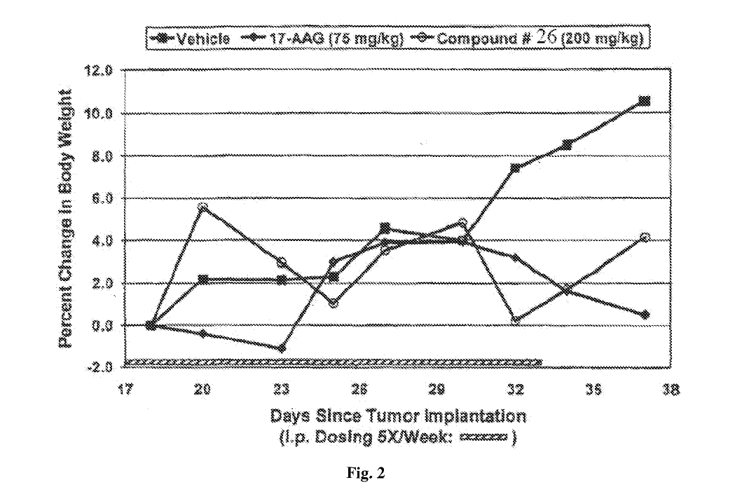

As shown in FIG. 1, treatment with 200 mg/kg body weight of Compound 26 decreased the growth rate of RERF-LC-A^{IVP} human lung tumor cells in nude mice, as did a dose of 75 mg/kg body weight of 17-AAG (an unrelated HSP90 inhibitor). This effect was not associated with overt toxicity, as shown by the minimal effect on body weights depicted in FIG. 2.

### Example 2

### The Combination of Compound 1 and Bevacizumab Displayed Enhanced Anti-Tumor Activity against Human Tumor Cells in SCID Mouse Xenograft Models

The human non-small cell lung cancer (NSCLC) cell line, NCI-H1975 (ATCC #CRL-5908) was obtained from the American Type Culture Collection (ATCC; Manassas, Va., USA). The human multiple myeloma cell line, RPMI 8226 (ATCC #CCL-155) was also obtained from the ATCC.

NCI-H1975 cells were cultured in growth media prepared from 50% Dulbecco's Modified Eagle Medium (high glucose), 50% RPMI Media 1640 (4.5 g/L glucose), 10% fetal bovine serum (FBS), 10 mM HEPES, 1% 100× Penicillin-Streptomycin, 1% 100× sodium pyruvate and 1% 100× MEM non-essential amino acids. RPMI 8226 cells were cultured in growth media prepared with RPMI Media 1640 (4.5 g/L glucose), 10% FBS, 10 mM HEPES, 1% 100× Penicillin-Streptomycin, 1% 100× sodium pyruvate and 1% 100× MEM non-essential amino acids. FBS was obtained from ATCC and all other reagents were obtained from Invitrogen Corp. (Carlsbad, Calif., USA). Cells that had been cryopreserved in liquid nitrogen were rapidly thawed at 37° C. and transferred to a tissue culture flask containing growth media and then incubated at 37° C. in a 5% CO₂ incubator. To expand the RPMI 8226 cell line, growth media was changed every 2-3 days and cultures were passaged 1:3 to 1:5 every 3-5 days. When the a 175 cm² flask reached approximately 20-40×10(6) total cells, the above passaging process was repeated until sufficient cells had been obtained for implantation into mice. To expand the NCI-H1975 cell line, cultures were split 1:5 every 3 days when 175 cm² flasks became 85% confluent. Cultures were passaged by washing with 10 mL of room temperature phosphate buffered saline (PBS) and then disassociating cells by adding 5 mL 1× trypsin-EDTA and incubating at 37° C. until the cells detached from the surface of the flask. To inactivate the trypsin, 5 mL of growth media was added and then the contents of the flask were centrifuged to pellet the cells. The supernatant was aspirated and the cell pellet was resuspended in 10 mL of growth media and the cell number determined using a hemocytometer. Cells were seeded into 175 cm² flasks containing 50 mL of growth media and incubated at 37° C. in a 5% CO₂ incubator. When the flasks reached 85% confluence, the above passaging process was repeated until sufficient cells had been obtained for implantation into mice.

Six to eight week old, female CB17/Icr-Prkdc^{scid}/Crl (SCID) mice were obtained from Charles River Laboratories (Wilmington, Mass., USA) Animals were housed 4-5/cage in micro-isolators, with a 12 hr/12 hr light/dark cycle, acclimated for at least 1 week prior to use and fed normal laboratory chow ad libitum. Animals were between seven to thirteen weeks of age at implantation. To implant NCI-H1975 or RPMI 8226 tumor cells into SCID mice, cells were collected as described above, washed in PBS and resusupended at a concentration of 5×10(7) cells/mL in 50% non-supplemented medium and 50% Matrigel Basement Membrane Matrix (#354234; BD Biosciences; Bedford, Mass., USA). Using a 27 gauge needle and 1 cc syringe, 5×10(6) NO-H1975 or RPMI 8226 cells in 0.1 mL of a cell suspension were injected subcutaneously into the flanks of SCID mice.

For the NCI-H1975 model, tumors were then permitted to develop in vivo until the majority reached approximately 100-225 mm³ in tumor volume, which required ˜1½ weeks. For the RPMI 8226 model, tumors were permitted to develop in vivo until the majority reached approximately 80-200 mm³ in tumor volume, which required ˜3½ weeks. Animals with oblong, very small or large tumors were discarded and only animals carrying tumors that displayed consistent growth rates were selected for studies. Tumor volumes (V) were calculated by caliper measurement of the width (W), length (L) and thickness (T) of tumors using the following formula: V=0.5236×(L×W×T). Animals were randomized into treatment groups so that the average tumor volumes of each group were similar at the start of dosing. % T/C values, as a measure of efficacy, were determined as follows:
(i) If ΔT>0: % T/C=(ΔT/ΔC)×100
(ii) If ΔT<0: % T/C=(ΔT/T₀)×100
(iii) ΔT=Change in average tumor volume between start of dosing and the end of study.
(iv) ΔC=Change in average tumor volume between start of dosing and the end of study.
(v) T₀=Average tumor volume at start of dosing.

To formulate the test article, Compound 1, in DRD, stock solutions of the test article were prepared by dissolving the appropriate amounts of the compound in dimethyl sulfoxide (DMSO) by sonication in an ultrasonic water bath. Stock solutions were prepared weekly, stored at −20° C. and diluted fresh each day for dosing. A solution of 20% Cremophore RH40 (polyoxyl 40 hydrogenated castor oil; BASF Corp., Aktiengesellschaft, Ludwigshafen, Germany) in 5% dextrose in water (Abbott Laboratories, North Chicago, Ill., USA) was also prepared by first heating 100% Cremophore RH40 at 50-60° C. until liquefied and clear, diluting 1:5 with 100% D5W, reheating again until clear and then mixing well. This solution can be stored at room temperature for up to 3 months prior to use. To prepare DRD formulations for daily dosing, DMSO stock solutions were diluted 1:10 with 20% Cremophore RH40. The final DRD formulation for dosing contained 10% DMSO, 18% Cremophore RH40, 3.6% dextrose, 68.4% water and the appropriate amount of test article. Animals were intravenous (i.v.) injected via the tail vein with this formulation at 10 mL per kg body weight on either 1 day each week or 5 days each week (Monday, Tuesday, Wednesday, Thursday, Friday). Bevacizumab (Avastin) at a concentration of 25 mg/mL was purchased from Genentech (South San Francisco, Calif., USA), stored at 4° C., and the appropriate amount of the test article was diluted fresh each day into 0.9% NaCl₂ saline Animals were intraperitoneal (i.p.) injected with this formulation at 10 mL per kg body weight on either 1 day each week or 2 days each week (Tuesday, Friday).

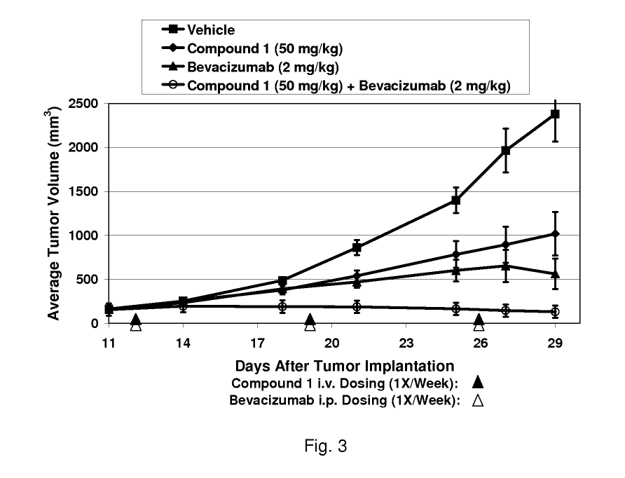

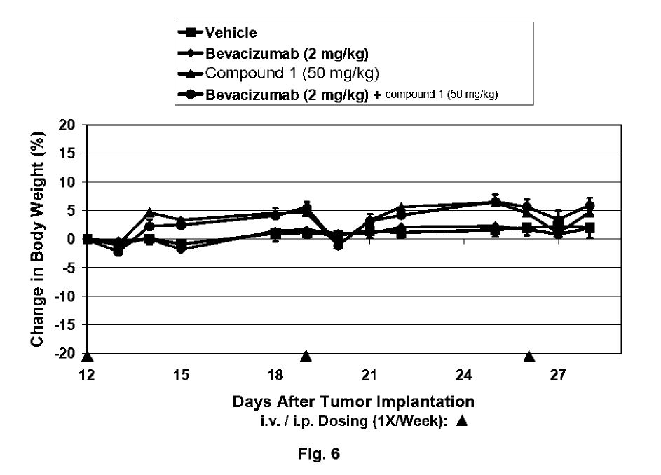

The combination of Compound 1 and bevacizumab was examined in the human NCI-H1975 NSCLC xenograft model. As shown in FIG. 3, treatment with a dose of 50 mg/kg body weight of Compound 1 on a once per week schedule moderately inhibited NCI-H1975 tumor growth in SCID mice, with a %T/C value of 39 observed on day 29 Similarly, treatment with a dose of 2 mg/kg body weight of bevacizumab on a once per week schedule moderately inhibited NCI-H1975 tumor growth in SCID mice, with a % T/C value of 18 observed on day 29. In contrast, concurrent treatment with a combination of 50 mg/kg body weight of Compound 1 plus 2 mg/kg body weight bevacizumab on a once per week schedule dramatically inhibited tumor growth and induced tumor regression, with a % T/C value of −17 observed on day 29. The efficacy observed for the combination treatment group was significantly greater than that observed for either single-agent group alone (P<0.05; one-way ANOVA). This effect was not associated with excessive toxicity, as the Compound 1 plus bevacizumab combination treatment group had an average bodyweight change on day 29 (last day measured) relative to the start of the study of +4.8% (+/−1.3 SEM), as compared to +2.0% (+/−1.9 SEM) for the vehicle-treated group (FIG. 6). Similar results were also observed combining Compound 1 with a lower 1.5 mg/kg body weight bevacizumab dose on a once per week schedule in this model (data not shown).

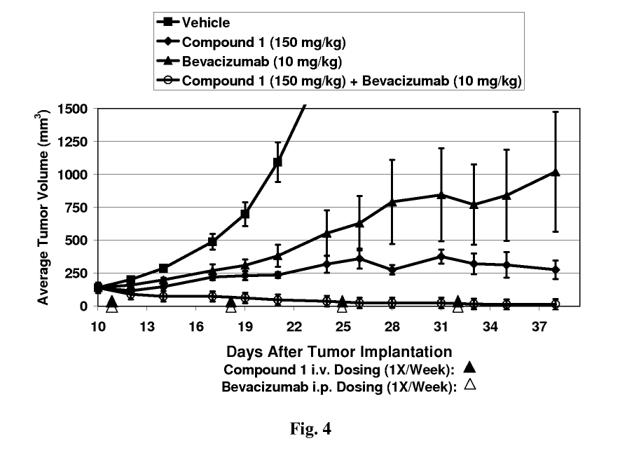

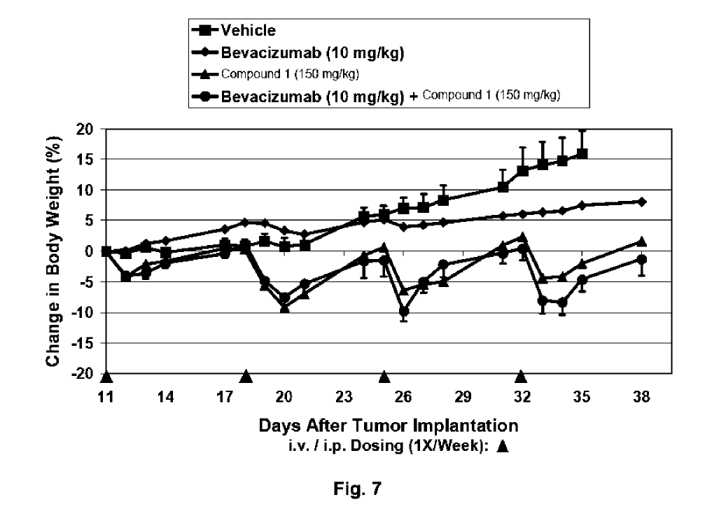

The combination of Compound 1 and bevacizumab was also examined in the NCI-H1975 model using higher doses of each test article. The highest non-severely toxic dose (HNSTD) of Compound 1 was found to be 150 mg/kg i.v. dosed once per week in tumor-bearing SCID mice (data not shown). The HNSTD was determined based on prior studies and was defined as the dose and dosing schedule at which no morbidity was observed, and at which no individual animal lost ≧20% of its body weight and no treatment group lost ≧10% average body weight over the course of the study. As shown in FIG. 4, treatment with a dose of 150 mg/kg body weight of Compound 1 on a once per week schedule substantially inhibited NCI-H1975 tumor growth in SCID mice, with a % T/C value of 3 observed on day 38. Treatment with a dose of 10 mg/kg body weight of bevacizumab on a once per week schedule moderately inhibited NCI-H1975 tumor growth in SCID mice, with a % T/C value of 17 observed on day 38. In contrast, concurrent treatment with a combination of 150 mg/kg body weight of Compound 1 plus 10 mg/kg body weight bevacizumab on a once per week schedule dramatically inhibited tumor growth and induced regressions in 100% of tumors, with a %T/C value of −90 observed on day 38. The efficacy observed for the combination treatment group was significantly greater than that observed for either single-agent group alone (P<0.05; one-way ANOVA). The combination treatment also caused 2 of 8 tumors to completely disappear by day 38, whereas no complete tumor responses were observed in any other treatment group. As shown in FIG. 7, this effect was not associated with excessive toxicity, as the Compound 1 plus bevacizumab combination treatment group had an average bodyweight change on day 38 relative to the start of the study of −1.3% (+/−2.7 SEM), as compared to +15.9% (+/−3.8 SEM) for the vehicle-treated group on day 35 (the vehicle group was terminated early on day 35 due to excessive tumor burden, which also resulted in increased body weight relative to the other groups).

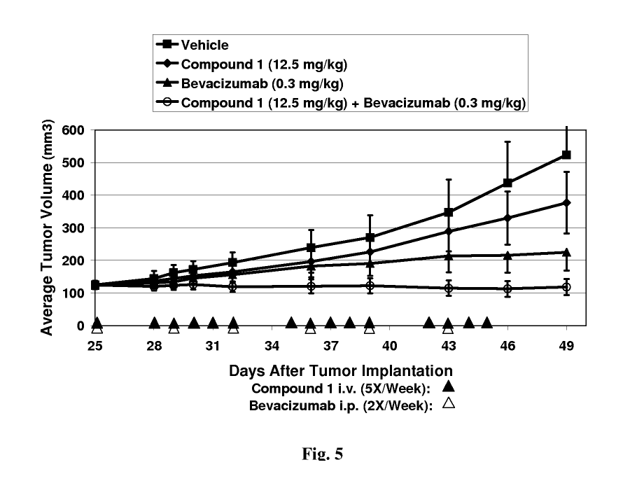

The combination of Compound 1 and bevacizumab was also examined in the human RPMI 8226 multiple myeloma xenograft model. As shown in FIG. 5, treatment with a dose of 12.5 mg/kg body weight of Compound 1 on a five times per week schedule moderately inhibited RPMI 8226 tumor growth in SCID mice, with a % T/C value of 63 observed on day 49. Treatment with a dose of 0.3 mg/kg body weight of bevacizumab on a twice per week schedule moderately inhibited RPMI 8226 tumor growth in SCID mice, with a % T/C value of 26 observed on day 49. In contrast, concurrent treatment with a combination of 12.5 mg/kg body weight of Compound 1 on a five times per week schedule plus 0.3 mg/kg body weight bevacizumab on a twice per week schedule dramatically inhibited tumor growth and induced slight tumor regression, with a % T/C value of −5 observed on day 49. The efficacy observed for the combination treatment group was significantly greater than that observed for either single-agent group alone (P<0.05; one-way ANOVA). This effect was not associated with excessive toxicity, as the Compound 1 plus bevacizumab combination treatment group had an average bodyweight change on day 38 relative to the start of the study of +3.4% (+/−1.1 SEM), as compared to +5.7% (+/−1.5 SEM) for the vehicle-treated group on day 49.

In a second study in the RPMI 8226 model, a single combination dose of 12.5 mg/kg Compound 1 and 1 mg/kg bevacizumab dramatically inhibited tumor growth over three days relative to single agents alone, with %T/C values of 83, 42 and −1 for the Compound 1 alone, bevacizumab alone and combination groups, respectively (data not shown).

In conclusion, the combination of Compound 1 and bevacizumab displayed greater efficacy than either single agent alone. Combination treatments did not result in additional toxicity relative to the single agents, with only minimal effects on cumulative average body weight changes over the course of the study.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A pharmaceutical combination comprising an VEGF inhibitor and an Hsp90 inhibitor according to the following formulae: 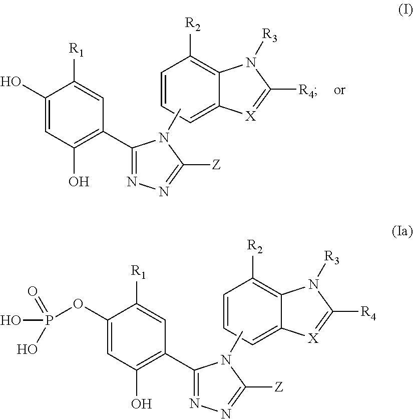
or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NRONR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNIZ₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —S(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

2. The combination of claim 1, wherein the Hsp90 inhibitor is selected from the group consisting of:
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole, and
3-(2,4-dihydroxy-5-isopropyl-phenyl)-4-(1-propyl-indol-5-yl)-5-mercapto-[1,2,4]triazole,
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate,
or a tautomer, or a pharmaceutically acceptable salt thereof.

3. The combination of claim 1, wherein the Hsp90 inhibitor is selected from the group consisting of:
3-(2,4-Dihydroxy-5-ethyl-phenyl)-4-(1-isopropyl-7-methoxy-indol-4-yl)-5-mercapto-[1,2,4]triazole;
3-(2,4-dihydroxy-5-isopropyl-phenyl)-4-(N-methyl-indol-5-yl)-5-mercapto-[1,2,4]triazole;
3-(2,4-dihydroxy-5-isopropyl-phenyl)-4-(1-methyl-indol-5-yl)-5-hydroxy-[1,2,4]triazole;
or a tautomer or pharmaceutically acceptable salt thereof.

4. The combination of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

5. The combination of claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

6. The combination of claim 4, wherein the VEGF inhibitor is bevacizumab, sunitinib, or sorafenib.

7. The combination according to claim 6, wherein the VEGF inhibitor is bevacizumab.

8. The combination according to claim 1, wherein the Hsp90 inhibitor is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof, and the VEGF inhibitor is bevacizumab.

9. The combination according to claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the VEGF inhibitor is bevacizumab.

10. A method of treating or preventing a proliferative disorder in a subject, comprising administering to a subject an effective amount of the combination of claim 1.

11. The method of claim 10, wherein the proliferative disorder is cancer.

12. The method of claim 11, wherein the cancer is associated with VEGF.

13. The method of claim 11, wherein the cancer is metastatic colorectal cancer, metastatic or non-metastatic breast cancer, non-small cell lung cancer, renal cell carcinoma, pancreatic cancer, ovarian cancer, hormone refractory prostate cancer, glioblastoma multiforme, non-metastatic unresectable liver cancer, gliosarcoma, malignant glioma, peritoneal cancer, fallopian tube cancer, rectal cancer, kidney cancer, Hodgkin's lymphoma, colorectal cancer, neuroendocrine carcinoma, bladder cancer, uveal melanoma, hormone sensitive breast cancer, hepatocellular cancer, gastric cancer, squamous cell carcinoma, cervical cancer, uterine cancer, chronic lymphocytic leukemia, lymphoma, myeloma, Karposi's sarcoma, urothelial carcinoma, mesothelioma, malignant fibrous histiocytoma or leiomyosarcoma.

14. The method of claim 13, wherein the cancer is metastatic colorectal cancer, metastatic or non-metastatic breast cancer, metastatic non-small cell lung cancer, renal cell carcinoma, metastatic or unresectable locally advanced pancreatic cancer, ovarian cancer, hormone refractory prostate cancer, glioblastoma multiforme, or non-metastatic unresectable liver cancer.

15. The method of claim 14, wherein the cancer is metastatic non-small cell lung cancer.

16. The method of claim 14, wherein the cancer is metastatic breast cancer.

17. The method of claim 14, wherein the cancer is metastatic colorectal cancer.

18. The method of claim 14, wherein the cancer is metastatic glioblastoma multiforme.

19. The method of any one of claim 10, wherein the subject is human.

20. A method of inhibiting the growth of a cancer or tumor cell in a subject, the method comprising the steps of: (a) contacting the cell with an effective amount of a compound of formulae (I) or (Ia) as defined in claim 1, and (b) exposing the cell to an effective amount of a VEGF inhibitor, wherein the VEGF inhibitor is selected from the group consisting of bevacizumab, sunitinib, or sorafenib.

21. The method of claim 20, wherein the compound is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof and the VEGF inhibitor is bevacizumab.

22. The method of claim 20, wherein the compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the VEGF inhibitor is bevacizumab.

23. A method of treating a subject with cancer. comprising the steps of: administering to the subject an effective amount of a compound of formulae (I) or (Ia) as defined in claim 1, and (b) admistering to the subject an effect amount of a VEGF inhibitor, wherein the VEGF inhibitor is selected from the group consisting of bevacizumab, sunitinib, or sorafenib.

24. The method of claim 23, wherein the compound is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof and the VEGF inhibitor is bevacizumab.

25. The method of claim 23, wherein the compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the VEGF inhibitor is bevacizumab.
